SEQUENCE LISTING (22 sequences)

SEQ ID NO: 1 - DNA, 44 bp, Artificial Sequence
  chemically synthesized
gacttatcagagccagcattgaggaatgagccaagtccaatggg

SEQ ID NO: 2 - DNA, 44 bp, Artificial Sequence
  chemically synthesized
cccattggacttggctcattcctcaatgctggctctgataagtc

SEQ ID NO: 3 - DNA, 45 bp, Artificial Sequence
  chemically synthesized
ggagtttgtcttcaccatgtggagaaaggagcattcaggcagagg

SEQ ID NO: 4 - DNA, 45 bp, Artificial Sequence
  chemically synthesized
cctctgcctgaatgctcctttctccacatggtgaagacaaactcc

SEQ ID NO: 5 - DNA, 41 bp, Artificial Sequence
  chemically synthesized
cacctcagtaagtgcaatttccaaaaacatccagggaaatc

SEQ ID NO: 6 - DNA, 41 bp, Artificial Sequence
  chemically synthesized
gatttccctggatgtttttggaaattgcacttactgaggtg

SEQ ID NO: 7 - DNA, 41 bp, Artificial Sequence
  chemically synthesized
gggtaccagccagcattttcatcatctaagggtcaggtgcc

SEQ ID NO: 8 - DNA, 41 bp, Artificial Sequence
  chemically synthesized
ggcacctgacccttagatgatgaaaatgctggctggtaccc

SEQ ID NO: 9 - DNA, 44 bp, Artificial Synthesized
  chemically synthesized
gacttatcagagccagcagatatcaatgagccaagtccaatggg

SEQ ID NO: 10 - DNA, 44 bp, Artificial Sequence
  chemically synthesized
cccattggacttggctcattgatatctgctggctctgataagtc

SEQ ID NO: 11 - DNA, 45 bp, Artificial Sequence
  chemically synthesized
ggagtttgtcttcaccatgaagcttaaggagcattcaggcagagg

SEQ ID NO: 12 - DNA, 45 bp, Artificial Sequence
  chemically synthesized
cctctgcctgaatgctccttaagcttcatggtgaagacaaactcc

SEQ ID NO: 13 - DNA, 41 bp, Artificial Sequence
  chemically synthesized
cacctcagtaagtgcaatttcgaatcccatccagggaaatc

SEQ ID NO: 14 - DNA, 41 bp, Artificial Sequence
  chemically synthesized
gatttccctggatgggattcgaaattgcacttactgaggtg

SEQ ID NO: 15 - DNA, 41 bp, Artificial Sequence
  chemically synthesized
gggtaccagccagcaagcttatcatctaagggtcaggtgcc

SEQ ID NO: 16 - DNA, 41 bp, Artificial Sequence
  chemically synthesized
ggcacctgacccttagatgataagcttgctggctggtaccc

SEQ ID NO: 17 - DNA, 27 bp, Artificial Sequence
  chemically synthesized
caccatggaagtggccaacttctacta

SEQ ID NO: 18 - DNA, 24 bp, Artificial Sequence
  chemically synthesized
caccatggcggcgggcttcccgta

SEQ ID NO: 19 - DNA, 24 bp, Artificial Sequence
  chemically synthesized
ctagcagtggccggaggaggcgag

SEQ ID NO: 20 - DNA, 27 bp, Artificial Sequence
  chemically synthesized
gtggagaatgaatggatggcaaatgac

SEQ ID NO: 21 - DNA, 23 bp, Artificial Sequence
  chemically synthesized
ccgagaggacccaggcaaacttt

SEQ ID NO: 22 - DNA, 1355 bp, Homo sapiens
gtggagaatgaatggatggcaaatgacttatcagagccagcattgaggaatgagccaagtccaatgggaacacaggaaaaggaccctttgctctgccaggagagtcacagaaggctttgctggtggaggatgagatgcaatttcacctgaaatgatgaggagtttgtcttcaccatgtggagaaaggagcattcaggcagagggaccagtctgagcaaaggcattcgggtgtaaaattattgcctaggattggagtgaaggatatgtagcgctactgacaagagactcctcttcaagagcaggccacatatcctttatttcccaggggtggaaacgggcaacctcacagaggaattgaatcataatgaattgaagaaagaaaaatctcctgaagttggcattataattaataggcttaggctcatcctactagtcctcacttagactcattctcatgtagaaacaaaacgtgattaatcacctcagtaagtgcaatttccaaaaacatccagggaaatctagggtccaggtgcaggacctttccaatctgagttggattttgcctccaggagagagcatgggtaccagccagcattttcatcatctaagggtcaggtgccttgaagtgtctgctggcacccagggaagagccccccagcttgctgtcacaggacacagctgctcccttcctggccaggtagcctcgtgccctcccagaatggacttgaagctggccgggctgcttgggggctgcagaggaagaagggggctgccggcaaacctgctgactcaggctccacgagggacgaagtaacactggactcctttcggcactccgagaatggggtgggggcgtcttcaaaggatttccctcccttcccagtgcttgtccctgctctcggtccgttttctgctaagatttggggattttcaggcctggaggaaagtcccttgggacgatcatagagcgctagcatgaatcacctgagagcgcggaaggaaagggtcggtcccagaggcgcggggaaggcagggtgggcggactaggagcccgcgctcgtttgctgaaggcttggaagtgcagcgcagaagacagagggtgactaggaagacgccgagcggggctgcgggccggcgggtgggggaggggaggcgggggaaggcggctgagttgggcctctggattgtgtgtgtctgtgtcagtgtgtgtgtgtgtgtgtgtctatgtgtgtgttcggcgcctggccagggtgatttcccataaaccacatgccccccagtcctctcttaaaagctgccgagggctggccgaggctcggcccggggaaagtgaaagtttgcctgggtcctctcgg

(19) United States
(12) Patent Application Publication
(10) Pub. No.: US 2009/0117155 A1
(43) Pub. Date: May 7, 2009

(54) TREATMENT OF HIV-1 BY MODULATION OF VPR ACTIVATION OF THE M-CSF PROMOTER

(76) Inventors: Jay Rappaport, Somers Point, NJ (US); Valerie Haine, Namur (BE); Tracy Fischer-Smith, Horsham, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 12/302,189

(22) PCT Filed: May 24, 2007
(86) PCT No.: PCT/US2007/069659

§ 371 Date: Nov. 24, 2008

Related U.S. Application Data
(60) Provisional application No. 60/809,013, filed on May 26, 2006.
(60) Provisional application No. 60/889,391, filed on Feb. 12, 2007.

(51) Int. Cl. A61K 39/12; C12N 15/11; C12N 15/00; C12N 5/06; C12N 1/21; A61K 31/575; C12Q 1/68; C12Q 1/70; C12Q 1/66; A61K 31/7105

(52) U.S. Cl. 424/208.1; 536/23.2; 435/320.1; 435/325; 435/252.3; 435/6; 435/5; 435/8; 514/44; 514/178

(57) ABSTRACT

Macrophage colony-stimulating factor (M-CSF) is important for human immunodeficiency virus-type 1 (HIV-1) infection, replication and survival of infected cells. The mechanism(s) by which HIV-1 infection increases M-CSF production are, however, poorly understood. Here, we report that HIV-1 Vpr enhances M-CSF promoter activity and production in primary human monocytes and macrophages. Vpr activates M-CSF transcription through four C/EBP beta binding sites present within the M-CSF promoter, possibly through increased phosphorylation of C/EBP beta. RU486 (mifepristone) blocked Vpr-mediated up-regulation of M-CSF, suggesting that Vpr activates M-CSF promoter activity via the glucocorticoid pathway. The invention provides new avenues for therapeutic interventions in HIV-1 infection and other diseases involving M-CSF dysregulation (including malignancy, osteoporosis, autoimmune disorders, arthritis, and obesity) using glucocorticoid antagonists and modulators of C/EBP beta activity.

### CROSS-REFERENCE TO RELATED APPLICATIONS

This application claims benefit under 35 USC 119(e) to U.S. Patent Application No. 60/809,013, filed May 26, 2006, and U.S. Patent Application No. 60/889,391, filed Feb. 12, 2007, the entire disclosures of which are hereby incorporated by reference in their entireties.

### STATEMENT REGARDING FEDERALLY SPONSORED RESEARCH OR DEVELOPMENT

The invention described herein was supported in part by NIH/NINDS grant 1RO1 NS047031. The United States government has certain rights in the invention.

### BACKGROUND OF THE INVENTION

1. Field of Invention

This invention relates to treatment of HIV INFECTION. The invention provides that HIV-1 Vpr up-regulates M-CSF in primary human macrophages by a mechanism involving C/EBPβ transcription factors. Interestingly, the glucocorticoid antagonist RU486 was able to inhibit the M-CSF activation mediated by Vpr. Furthermore, dexamethasone was also shown to activate M-CSF transcription in macrophages, thus M-CSF activation in primary human macrophages is mediated via the glucorticoid pathway. Since M-CSF is linked to the glucocorticoid pathway, drugs like RU-486 are beneficial in HIV Infection. Such treatments may also be beneficial in osteoporosis, obesity, lipodystrophy and other diseases.

2. Description of Related Art

Macrophage colony-stimulating factor (M-CSF or CSF-1) is a growth factor controlling the survival, proliferation, differentiation, and other functions of mononuclear phagocytes (MPs). M-CSF exerts its activities by binding to a high-affinity receptor tyrosine-kinase encoded by the c-fms proto-oncogene, which leads to autophosphorylation of the receptor and triggers a series of phosphorylation cascades (Pixley and Stanley, 2004).

M-CSF has been demonstrated to play a significant role in human immunodeficiency virus-type 1 (HIV-1) infection in cells of the monocyte/macrophage lineage. M-CSF enhances the susceptibility of uninfected macrophages to HIV-1 infection and virus replication by up-regulating cell surface receptors involved in viral entry, CD4 and CCR5 (Bergamini et al., 1994; Oravecz et al., 1997). In macrophages that are HIV-1 infected, M-CSF may contribute to the maintenance of the viral reservoir by promoting the survival of infected cells (Gruber et al., 1995; Kalter et al., 1991). M-CSF also promotes differentiation of monocytes toward a subset of macrophages that are more susceptible to HIV infection. M-CSF derived macrophages express high levels of a large isoform of CCAAT/enhancer binding protein β (C/EBPβ), liver enriched transcriptional activator protein (LAP) (Komuro et al., 2003). Interestingly, C/EBP sites in the HIV LTR are necessary for virus replication in macrophages but not T cells (Henderson and Calame, 1997).

M-CSF may also contribute to AIDS pathogenesis through a variety of mechanisms including increased infection, survival and alterations in myeloid homeostasis and trafficking (Haine et al., 2006). Monocytes treated with rhM-CSF up-regulate CD16 (Ji et al., 2000) and plasma M-CSF levels correlate significantly with CD14+CD16+ monocytes in patients with chronic renal failure undergoing dialysis (Saionji and Ohsaka, 2001). Phenotypically similar cells are expanded in circulation of patients with HIV-1 associated dementia (HIV-D) (Pulliam et al., 1997) and accumulate in the central nervous system (CNS) perivascularly and within the brain parenchyma of patients with HIV encephalopathy (HIVE) (Fischer-Smith et al., 2001), the pathology of HIV-D. Interestingly, M-CSF levels are elevated in the cerebrospinal fluid (CSF) in HIV-1-infected individuals with HIV-D (Gallo et al., 1994), potentially contributing to the trafficking and survival of infected and non-infected macrophages into the CNS(Meltzer et al., 1990). This activity may be mediated in part by the role of M-CSF in activating chemokine production (Si et al., 2002). Mice showing a null mutation in the M-CSF gene are protected from neuroinvasion by Listeria monocytogenes, emphasizing the role of M-CSF in facilitating entry of monocytes/macrophages in the CNS (Jin et al., 2002).

Macrophage colony-stimulating factor (M-CSF or CSF-I) appears to play an important role in promoting and maintaining macrophage reservoirs of human immunodeficiency virus type 1 (HIV-1) through its effects on monocyte proliferation, differentiation, susceptibility to infection and increased survival. M-CSF could also play a role in HIV-associated central nervous system disorders; M-CSF levels are moreover elevated in the cerebrospinal fluid in HIV-1 infected patients with dementia. The role of viral and cellular regulators on M-CSF regulation was determined by first cloning the M-CSF promoter upstream the luciferase reporter gene. We determined the role of Vpr, Tat and Rev as well as C/EBPβ in THPI cells for M-CSF promoter enhancement by cotransfection. Both Vpr and C/EBPβ upregulate M-CSF promoter activity in this cell line. In primary human monocytes, Vpr also upregulates M-CSF promoter activity. ELISA assay of culture supernatants demonstrated a 8-fold increase in M-CSF production in response to ectopically expressed Vpr. In view of the importance of M-CSF in monocyte/macrophage differentiation and survival, the role of Vpr in M-CSF activation may be an important aspect in the pathogenesis of HIV infection, the maintenance of the macrophage/microglial reservoir of HIV infection, and the development of CNS manifestations of AIDS. Several studies have demonstrated that HIV-1 infection increases M-CSF production in macrophages; however the mechanism(s) had not been previously investigated. Here, we show that HIV-1 Vpr up-regulates M-CSF promoter activity and secreted M-CSF in primary human monocytes and macrophages. Results suggest that all four C/EBPβ binding sites identified in the M-CSF promoter are important for Vpr-dependent M-CSF transcription. We also show that C/EBPβ binds directly to the M-CSF promoter and activates M-CSF transcription and secretion in primary cells. Interestingly, our results indicate that Vpr increases C/EBPβ phosphorylation but not expression in primary macrophages. C/EBPβ activity has been shown to be regulated by phosphorylation and several phosphorylation sites have been identified in C/EBPβ (Akira, 1997). Here, we used an antibody specific for threonine (Thr)-235, which is phosphorylated by a ras-dependent mitogen-activated protein (MAP) kinase cascade (Nakajima et al., 1993). Our observations are in contrast to the findings of Roux et al, who proposed that Vpr enhances C/EBPβ expression after observing an increase in binding intensity of the C/EBPβ complex to the IL-8 promoter in Vpr-transfected A549 cells (Roux et al., 2000). This may suggest that the role of Vpr on C/EBPβ regulation is cell-dependent.

Approximately 30% of individuals infected with HIV-1 develop complications of the central nervous system (CNS), including motor disturbances, cognitive impairments and behavioral changes. It has been proposed that HIV-1 enters the brain by the infiltration of infected monocytes, which later differentiate into macrophages. The virus can replicate in these cells and spread to other cells of the brain, such as microglia, the brain-resident macrophages. The survival, proliferation and differentiation of the monocyte/macrophage lineage are controlled by macrophage colony-stimulating factor (M-CSF). M-CSF plays a crucial role in HIV-I infection of macrophages, resulting in a positive feedback loop where HIV-1 increases M-CSF production (and thus, promoting the survival of infected macrophages) which in turn enhances the susceptibility of macrophages to HIV-1 infection (FIG. 1). M-CSF could play a role in HIV-associated CNS disorders. Indeed, M-CSF levels are elevated in the cerebrospinal fluid (CSF) in HIV-infected patients with dementia. Interestingly, mice showing a null mutation in the M-CSF gene are protected from neuroinvasion by Listeria monocytogenes, suggesting a role for M-CSF in facilitating entry of monocytes/macrophages in the CNS2. The mechanism(s) by which HIV-I infection increase M-CSF production by macrophages were previously unknown. Here, the inventors show that HIV-I Vpr and the cellular factor C/EBPβ are involved in M-CSF activation in monocytes.

The Vpr-dependent effects on M-CSF are neutralized by the C/EBPβ inhibitor LIP or by a siRNA specific for C/EBPβ. We also show that the glucocorticoid antagonist RU486 was able to inhibit the M-CSF activation mediated by Vpr, suggesting that the inhibition of HIV-1 replication in primary macrophages by RU486 observed by Schafer et al. could be explained by a decrease in M-CSF (Schafer et al., 2006). It is worth noting that similar concentrations of RU486 were required to inhibit infection of monocytes/macrophages and to inhibit Vpr activation of M-CSF production.

The involvement of Vpr in HIV-1-associated neurological disorders has been suggested. Vpr has neurocytopathic effects on human neurons (Patel et al., 2002) and extracellular Vpr is found in the CSF of HIV-1 infected patients (Levy et al., 1994). Interestingly, M-CSF levels are elevated in HIV-1-infected individuals with dementia (Gallo et al., 1994). By up-regulating M-CSF, Vpr could also contribute to other HIV-1-associated diseases potentially involving M-CSF such as HIV-1-associated nephropathy and osteoporosis (for review, see (Haine et al., 2006)). The central role of Vpr in M-CSF production by macrophages could explain the requirement of Vpr for HIV-1 replication in vivo. Rhesus macaques and chimpanzees infected with Vpr mutants showed a spontaneous mutant reversion to a parental phenotype (Goh et al., 1998; Lang et al., 1993). Interestingly, the selection for Vpr function in vivo also occurs in humans since a laboratory worker accidentally infected with the HIV-1IIIb strain (containing a frame shift mutation at codon 73, resulting in a truncated Vpr protein with only the first 72 amino acids) showed a reversion to an intact Vpr and at the same time induced a shift toward macrophage tropic virus (Goh et al., 1998). It is possible that macrophage infection is required prior to successful T cell tropic viral infection in order for HIV-1 induced M-CSF production in macrophages to promote the immunosuppressive macrophage phenotype and contribute to defects in DC populations.

M-CSF exerts important effects in altering myeloid differentiation pathways, which may contribute to immune suppression and osteoporosis in the context of HIV-1 infection. Monocytes/macrophages are heterogeneous populations with pleiotropic phenotypic and functional characteristics (For review, see (Mosser, 2003)). M-CSF promotes the differentiation of type-2 macrophages (MΦ-2), which inhibit Th1 responses and produce IL-10. IL-10 promotes cFMS (M-CSF receptor) expression by dendritic cells (DC) rendering them susceptible to the effects of M-CSF produced by the same DC or other cell types. The expression of cFMS promotes the conversion of immature DC to macrophages (Rieser et al., 1998) which could contribute to impairments in DC numbers and function in HIV-1 infection. M-CSF, together with receptor activator of nuclear factor kappa B ligand (RANKL) can differentiate monocytes into osteoclasts instead of macrophages (Khosla, 2001) and immature DC exposed to the same cytokines can be transdifferentiated to osteoclasts, as well (Rivollier et al., 2004). In view of the increased incidence of osteoporosis in HIV-1 infection (Annapoorna et al., 2004), the effects of virus-induced M-CSF could have important implications for loss of DC as well as the increase in osteoclasts and suppressive macrophages.

M-CSF is a powerful immunosuppressant with increased levels associated with immunosuppressive conditions including HIV-1 infection/AIDS (Gallo et al., 1994), pregnancy (Saito et al., 1992) and certain cancers (Kacinski, 1995). In fact, op/op female mice (which are defective in M-CSF) have reduced rates of successful pregnancy when mated to non-syngeneic males (Pollard et al., 1991). M-CSF appears to induce macrophages to become immunosuppressive (Wing et al., 1986). Furthermore, renal cell carcinoma as well as a panel of related tumor cells producing IL-6 and M-CSF (which we show here induces IL-6) inhibit DC differentiation from CD34+ stem cells (Menetrier-Caux et al., 1998). Increased M-CSF pretreatment levels have been associated with poor prognosis in non-small cell lung carcinoma (Kaminska et al., 2006). M-CSF, therefore, could be involved in the mechanism of both HIV-1 viral and tumor escape via the generation of suppressive macrophages and at the same time inhibiting the generation of DC.

In view of the above findings, a need exists for a manner of regulating M-CSF production, particularly in individuals who are infected with HIV-1. The present invention provides methods and compositions which may be utilized to help individuals with such conditions and provides further insight into the regulation of M-CSF gene expression.

All references cited herein are incorporated herein by reference in their entireties.

### BRIEF SUMMARY OF THE INVENTION

Despite the known activities of M-CSF in promoting HIV infection and AIDS pathogenesis, the mechanism(s) by which HIV-1 infection increases M-CSF production in infected macrophages has not been previously elucidated. The invention provides that HIV-1 Vpr up-regulates M-CSF in primary human macrophages by a mechanism involving C/EBPβ transcription factors. Interestingly, the glucocorticoid antagonist RU486 was able to inhibit the M-CSF activation mediated by Vpr. Furthermore, dexamethasone was also shown to activate M-CSF transcription in macrophages, thus M-CSF activation in primary human macrophages is mediated via the glucorticoid pathway.

M-CSF production is increased in HIV infection. This leads to increases in susceptible macrophages and an increased survival of macrophages. Since M-CSF derived macrophages and dendritic cells are associated with immune stimulatory properties, and since M-CSF derived macrophages have phagocytic and immune suppressive activities (producing IL-10 upon activation instead of IL-12), strategies that reduce M-CSF production would reduce virus production, reduce the reservoir of productive virus infection (infected tissue macrophages) and increase immune function.

M-CSF and RANKL are involved in the differentiation of monocytes to osteoclasts. It is therefore interesting that HIV patients not only have immune suppression, but a significant frequency of osteoporosis.

The inventors have demonstrated that M-CSF is linked to the glucocorticoid pathway and that drugs like RU-486 is beneficial in HIV Infection. Such treatments will also be beneficial in osteoporosis, obesity, lipodystrophy and other diseases.

In summary, the inventors demonstrated the central role of HIV-1 Vpr in M-CSF regulation. Vpr activates C/EBPβ factors by phosphorylation, which can then activate M-CSF transcription in the context of 4 C/EBPβ binding sites present in the M-CSF promoter. The inhibition of Vpr-dependent M-CSF activation by the glucocorticoid receptor antagonist RU486 as well a by a siRNA specific for C/EBPβ show that these strategies are useful for the treatment of HIV-1 infection/AIDS and other diseases involving M-CSF dysregulation.

The inventors have found that ectopic expression of HIV-1 Vpr in primary human monocytes upregulates M-CSF promoter activity and M-CSF production. The Vpr-dependent effects on M-CSF are neutralized by LIP, the short isoform of C/EBPβ. Taken together, these inventors have shown that HIV-1 Vpr upregulates M-CSF via a mechanism involving C/EBPβ factors. The M-CSF activation induced by Vpr may facilitate the establishment and maintenance of infected macrophages as a reservoir for HIV and may be involved in HIV-associated CNS disorders.

DHEA, a circulating adrenal steroid and available supplement, has been demonstrated to antagonize the action of glucocorticoids. There is some anecdotal evidence that DHEA may be beneficial in AIDS and RU-486 and DHEA should synergize. DHEA appears to work downstream of the glucocorticoid receptor whereas RU486 interacts with it directly and prevents its translocation to the nucleus.

The invention provides a nucleic acid comprising a reporter gene operatively linked to an M-CSF promoter region and the gene encoding HIV-1 Vpr. The invention further provides that the reporter gene is a luciferase gene. The invention further provides that the M-CSF promoter region is operatively linked to a CAAT enhancer binding protein site. The invention provides an isolated oligonucleotide comprising the M-CSF promoter region, wherein the M-CSF promoter region is responsive to C/EBPβ. The invention provides a reporter vector comprising the M-CSF promoter region, wherein the M-CSF promoter region is responsive to C/EBPβ. The invention provides a host cell comprising a reporter gene operatively linked to an M-CSF promoter region and the gene encoding HIV-1 Vpr, wherein the M-CSF promoter region is responsive to C/EBPβ. The invention further provides that the host cell is a member selected from the group consisting of monocyte, macrophages, THP-1 cells, eukaryotic cells, and prokaryotic cells. The invention provides a cell line stably transfected with a reporter vector comprising the M-CSF promoter, wherein the M-CSF promoter region is responsive to C/EBPβ.

The invention provides a method of identification of compounds that modulate M-CSF promoter activity comprising providing a reporter vector comprising a reporter gene and an M-CSF promoter region, wherein the M-CSF promoter region is responsive to C/EBPβ, providing a test agent, providing C/EBPβ, combining the reporter vector, the test agent, and C/EBPβ, measuring reporter gene activity in the presence of test agent, measuring reporter gene activity in a control sample and comparing reporter gene activity in the control sample compared to the test sample, to identify a compound which modulates M-CSF promoter activity. The invention further provides that the reporter vector comprising a reporter gene and the M-CSF promoter region responsive to C/EBPβ is in a stably transfected cell line.

The invention provides a method of identifying a compound for the treatment of HIV-infection comprises determining the effect of the compound on HIV-1 Vpr-induced activation of the M-CSF promoter.

The invention provides a method of identifying a potential HIV-1 therapeutic agent which inhibits Vpr-induced activation of the M-CSF promoter comprising the steps of, providing a cell comprising a reporter gene operatively linked to an M-CSF promoter region, contacting the cell with a test agent in the presence of HIV-1 Vpr, wherein a decrement in the expression of the reporter gene in the presence of HIV-1 Vpr and the test agent, as compared to the expression of the reporter gene in the presence of Vpr and the absence of the test agent, indicates that the test agent is a potential HIV-1 therapeutic.

The invention provides a method of identifying a potential HIV-1 therapeutic agent which inhibits HIV-1 Vpr-induced activation of the M-CSF promoter comprising the steps of providing a cell comprising a reporter gene operatively linked to an M-CSF promoter region, and a vector comprising the gene encoding HIV-1 Vpr, contacting said cell with a test agent under conditions wherein said cells express Vpr, wherein a decrement in the expression of the reporter gene as compared to a control indicates that the test agent is a potential HIV-1 therapeutic. The invention further provides that the cell is a member selected from the group consisting of monocytes, macrophages, THP-1 cells, eukaryotic cells, and prokaryotic cells. The invention further provides that the M-CSF promoter is operatively linked to a CAAT enhancer binding protein site. The invention further provides that the reporter gene is a luciferase gene.

The invention provides a method of treating HIV infection in a patient in need thereof by administration of an effective amount of a glucocorticoid antagonist. The invention further provides that the glucocorticoid antagonist is administered in an amount sufficient to have an effect selected from the group consisting of decreasing M-CSF levels in plasma, decreasing M-CSF levels in cerebrospinal fluid, decreasing M-CSF production by macrophages, and combinations thereof. The invention further provides that the glucocorticoid antagonist is administered in combination with a compound selected from the group consisting of DHEA, antiretroviral drugs, M-CSF receptor antagonists, soluble M-CSF receptor molecules, and combinations thereof.

The invention provides a method of treating a disease selected from the group consisting of malignancy, osteoporosis, autoimmune disorders, arthritis, obesity, and lipodystrophy in a patient in a need thereof by administration of a glucocorticoid antagonist in an amount sufficient to have an effect selected from the group consisting of decreasing M-CSF levels in plasma, decreasing M-CSF levels in cerebrospinal fluid, decreasing M-CSF production by macrophages, and combinations thereof. The invention further provides that the glucocorticoid antagonist is administered in combination with a compound selected from the group consisting of DHEA, antiretroviral drugs, M-CSF receptor antagonists, soluble M-CSF receptor molecules, and combinations thereof. The invention further provides that the glucocorticoid antagonist is RU486.

The invention provides a method of treating HIV infection in a patient in a need thereof by administration of an siRNA targeting HIV-1 Vpr-induced activation of the M-CSF promoter administered in an amount sufficient to have an effect selected from the group consisting of decreasing M-CSF levels in plasma, decreasing M-CSF levels in cerebrospinal fluid, decreasing M-CSF production by macrophages, and combinations thereof.

The invention provides a method of treating HIV infection in a patient in a need thereof by administration of a glucocorticoid antagonist in an amount sufficient to have an effect selected from the group consisting of decreasing M-CSF levels in plasma, decreasing M-CSF levels in cerebrospinal fluid, decreasing M-CSF production by macrophages, and combinations thereof, in combination with an HIV antigen.

The invention provides a method of treating HIV infection in a patient in a need thereof by administration of a glucocorticoid antagonist in an amount sufficient to have an effect selected from the group consisting of decreasing M-CSF levels in plasma, decreasing M-CSF levels in cerebrospinal fluid, decreasing M-CSF production by macrophages, and combinations thereof, after administration of an HIV antigen.

The invention provides a method of preventing or treating HIV infection in a patient in a need thereof comprising administering a preventive or prophylactic HIV vaccine, after exposure to the preventive or prophylactic HIV vaccine, administering a glucocorticoid antagonist, whereby HIV infection is prevented or treated. The invention further provides that the glucocorticoid antagonist is RU486.

The invention provides a method of treating HIV infection in a patient in need thereof by administration of an effective amount of a compound which modulates activity of a member of the group selected from M-CSF promoter, M-CSF, Vpr, C/EBPβ_{..} and combinations thereof. The invention further provides that the compound which modulates activity of a member of the group selected from M-CSF promoter, M-CSF, Vpr, C/EBPβ_{..} and combinations thereof is administered in an amount sufficient to have an effect selected from the group consisting of decreasing M-CSF levels in plasma, decreasing M-CSF levels in cerebrospinal fluid, decreasing M-CSF production by macrophages, and combinations thereof. The invention further provides that the compound which modulates activity of a member of the group selected from M-CSF promoter, M-CSF, Vpr, C/EBPβ_{..} and combinations thereof is administered in combination with a compound selected from the group consisting of DHEA, antiretroviral drugs, M-CSF receptor antagonists, soluble M-CSF receptor molecules, and combinations thereof.

The invention provides a method of treating a disease selected from the group consisting of malignancy, osteoporosis, autoimmune disorders, arthritis, obesity, and lipodystrophy in a patient in a need thereof by administration of a compound which modulates activity of a member of the group selected from M-CSF promoter, M-CSF, Vpr, C/EBPβ_{..} and combinations thereof in an amount sufficient to have an effect selected from the group consisting of decreasing M-CSF levels in plasma, decreasing M-CSF levels in cerebrospinal fluid, decreasing M-CSF production by macrophages, and combinations thereof. The invention further provides that the compound which modulates activity of a member of the group selected from M-CSF promoter, M-CSF, Vpr, C/EBPβ_{..} and combinations thereof is administered in combination with a compound selected from the group consisting of DHEA, antiretroviral drugs, M-CSF receptor antagonists, soluble M-CSF receptor molecules, and combinations thereof. The invention further provides that the compound that modulates activity of a member of the group selected from M-CSF promoter, M-CSF, Vpr, C/EBPβ_{..} and combinations thereof is RU486.

The invention provides an assay kit comprising a reporter vector comprising the M-CSF promoter, wherein the M-CSF promoter is responsive to C/EBPβ.

### BRIEF DESCRIPTION OF SEVERAL VIEWS OF THE DRAWINGS

FIG. 1. Vpr Induction of the M-CSF Promoter. Macrophages produce high levels of M-CSF following entry and replication of HIV-1. This M-CSF production enhances the susceptibility of macrophages to HIV-1 infection, probably by upregulating the cell surface receptors involved in viral entry, CD4 and CCR55-6, and by upregulating transcription factors that act directly on the viral long terminal repeat (LTR). M-CSF was shown to induce the expression of the large isoform of C/EBPβ (LAP) in peripheral blood mononuclear cells7. C/EBPβ binding sites are present both in M-CSF promoter and HIV-1 L TR. LAP stimulates HIV-1 replication in macrophages. The short isoform of C/EBPβ, LIP (which lacks the trans activation domain) inhibits the transcriptional activity of LAP by forming heterodimers with LAP or by competing for DNA binding sites as a homodimer.

FIG. 2. The effect of HIV-1 proteins Vpr, Tat and Rev, and the large isoform of C/EBPβ, Liver-enriched transcriptional Activator Protein (LAP) on M-CSF promoter activity in THPI cells. The M-CSF promoter was cloned upstream the luciferase reporter gene of the pGL3basic vector (PMCSFpGL3). In the conditions tested, HIV-I Vpr and LAP upregulate M-CSF promoter activity by 3- and 7-fold, respectively; little or no effect is observed with HIV-I Tat or Rev.

FIG. 3. The effect of Vpr on M-CSF regulation in primary cells. Twenty-four hours after transfection, the M-CSF promoter activity and M-CSF production were analyzed (FIG. 3A). Transfection of primary macrophages indicated that the increase in M-CSF promoter activity and M-CSF production is Vpr-dependent (FIG. 3B).

FIG. 4. Primary human macrophages were transfected with pGL3-M-CSF construct along with HIV-1 molecular clone wild-type (pNL4.3) or mutated in the vpr gene (pNL4.3Δvpr) (FIG. 4A-4C). The amount of DNA was normalized with pcDNA plasmid. Twenty four hours after transfection, the M-CSF promoter activity, the M-CSF production and the IL-6 production were measured. The promoter activity is expressed in relative light unit (RLU) per μg of total protein. (*, p value<0.001). Co-transfection of Vpr with an siRNA specific for C/EBPβ decreased Vpr-dependent M-CSF production. Inhibition of C/EBPβ neutralizes Vpr-dependent M-CSF activation. Primary human macrophages were transfected with pGL3-M-CSF construct along with HIV-1 Vpr expression plasmid in the presence of a LIP expression vector or a siRNA targeting C/EBPβ. Twenty four hours after transfection, the promoter activity (FIG. 4D) and the M-CSF production (FIG. 4E) were measured (*,p value<0.001).

FIG. 5. The human M-CSF promoter region (SEQ ID NO: 22), with C/EBPβ biding sites underlined.

FIG. 6. Four putative C/EBPβ binding sites were identified in the human M-CSF promoter using TF search (threshold score of 84). (FIG. 6A) The binding sites of transcription factors previously shown to interact with HIV-1 LTR promoter (Pereira et al., 2000) are also found in the M-CSF promoter, including NFκB (2 sites), AP-1 (4 sites), Sp-1 (2 sites), Oct-1 (3 sites), USF (4 sites) and IRF (1 site). (FIG. 6B) Induction of M-CSF Promoter Activity.

FIG. 7. Vpr Induction of M-CSF Promoter Activity. Primary human monocytes CD14+ were transfected with pM-CSF luciferase reporter construct along with HIV-I Vpr expression plasmid in the presence or absence of a plasmid expressing LIP. The amount of DNA was normalized with pcDNA plasmid (empty vector). The luciferase activity (FIG. 7A) and the M-CSF production in the culture supernatants (FIG. 7B) were measured 24 hours post-transfection. The luciferase activity is expressed in relative light unit (RLU) per μg of total protein

FIG. 8. C/EBPβ Binding to the M-CSF Promoter. Electrophoretic mobility shift assays (EMSA) were performed using double-stranded biotin-labeled oligonucleotide probes containing each of the 4 putative C/EBPβ binding sites from the M-CSF promoter region (see Table III). Each of the four probes (Pmcsf_C/EBPβ1-4) are represented by panels A-D. Protein binding to each of the 4 C/EBPβ probes was abolished by an excess of unlabeled probes carrying the same sequence, demonstrating the specificity of the interaction (lanes 3 and 6, FIG. 8, Panels A-D). Addition of anti-C/EBPβ antibody to the binding mixture induced a supershift of the specific DNA-protein complexes, confirming that C/EBPβ was present in these complexes (lanes 4 and 7, FIG. 8, Panels A-D). The formation of the complex was sequence specific since no complex was observed with the probes corresponding to the mutated sequences within each of the 4 C/EBPβ binding sites, showing that the mutated nucleotides are essential for C/EBPβ binding on the M-CSF promoter (lane 8, FIG. 8, Panels A-D).

FIG. 9. siRNA efficiently decreases the level of C/EBPβ protein. Western blot analyses clearly demonstrated that the siRNA efficiently decreases the level of C/EBPβ protein.

FIG. 10. RU486 inhibits Vpr-dependent M-CSF activation. Primary human macrophages were co-transfected with pGL3-M-CSF and the Vpr expression vector and cultured with increasing concentrations of RU486. Cell lysates and supernatants collected at 1 or 2 days post-transfection showed a dose-dependent decrease in M-CSF promoter activity (FIG. 10A) and M-CSF production (FIG. 10B).

FIG. 11. The effect of dexamethasone on the M-CSF promoter. Primary macrophages transfected with the pGL3-M-CSF construct in the presence of different concentrations of dexamethasone show an increase in M-CSF promoter activity (FIG. 11A). In contrast, no increase was observed after addition of progesterone (FIG. 11B).

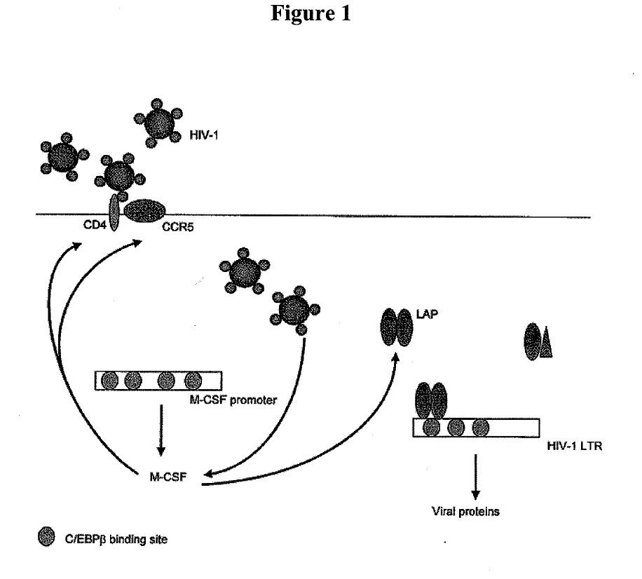

### DETAILED DESCRIPTION OF PREFERRED EMBODIMENTS OF THE INVENTION

HIV-1 Vpr Up-Regulates M-CSF

The human M-CSF promoter (bp −1313 to +48) was cloned upstream of the luciferase reporter gene in the pGL3 basic vector to generate pGL3-M-CSF. Regulation of the M-CSF promoter was first examined in the human monocytic leukemia cell line, THP1 (Table I). Co-transfection experiments using pGL3-M-CSF and HIV-1 Vpr, Tat or Rev expression plasmids demonstrated that HIV-1 Vpr up-regulates M-CSF promoter activity by 3-fold at 24 hours; no significant effect was observed with HIV-1 Tat or Rev.

TABLE I
TABLE I. M-CSF promoter activity following Vpr, Tat or Rev
expression in THP1 cells.

| Expression | Promoter activity |
|---|---|
| vector | (RLU/Ug prot) ± SD |
| Empty | 29.2 ± 5.6 [symbol] |
| Vpr | 98.4 ± 16.0 [symbol] |
| Tat | 43.0 ± 16.8 |
| Rev | 47.9 ± 14.4 |

[symbol]

, p value < 0.05

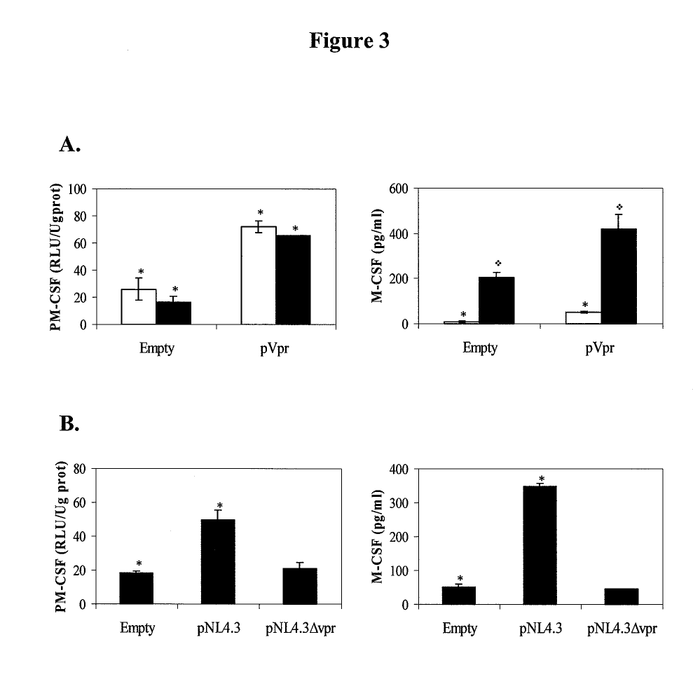

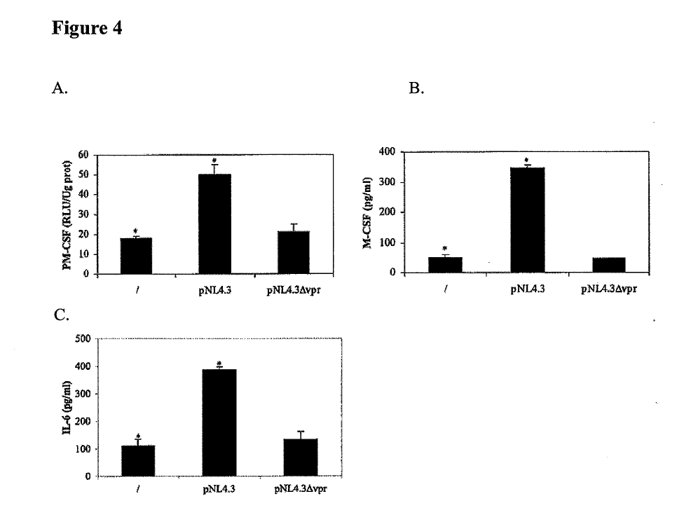

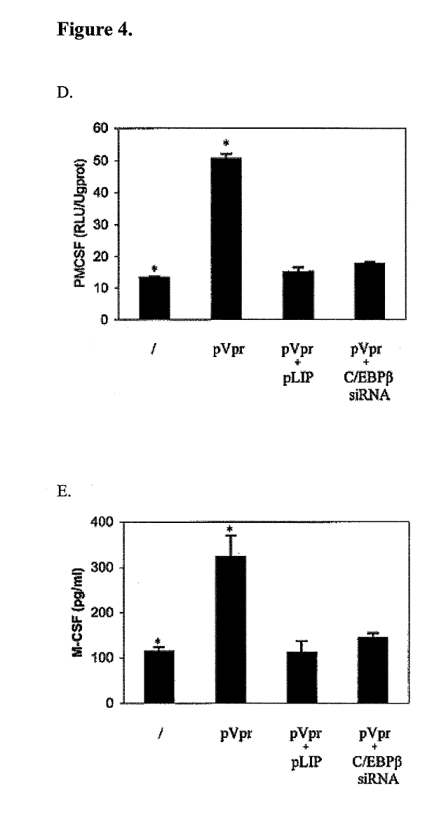

The effect of Vpr on M-CSF regulation was next examined in primary cells. Primary human monocytes or macrophages were transfected with pGL3-M-CSF and the Vpr expression vector. Twenty-four hours after transfection, the M-CSF promoter activity and M-CSF production were analyzed (FIG. 3A). Vpr was shown to stimulate the transcriptional activity of the M-CSF promoter in primary monocytes and macrophages 3- and 4-fold, respectively. ELISA assay of M-CSF in cell supernatants (reflecting production from the chromosomal M-CSF gene) demonstrated that Vpr increases M-CSF levels by 6-fold in primary monocytes and by 3-fold in primary macrophages. The importance of Vpr in M-CSF regulation was further demonstrated by comparison of the effects of the HIV-1 molecular clone pNL4.3 (Adachi et al., 1986) and its Vpr deleted derivative pNL4.3ΔVpr. Transfection of primary macrophages indicated that the increase in M-CSF promoter activity and M-CSF production is Vpr-dependent (FIG. 3B). Furthermore, co-transfection of both the Vpr defective provirus with wild-type Vpr did not result in increases in M-CSF promoter activity or production relative to the Vpr construct alone (data not shown), suggesting that Vpr is the major component of HIV-1 responsible for M-CSF production. In contrast to the wild-type provirus, the Vpr defective provirus failed to increase the production of IL-6 (data not shown) in agreement with results previously reported by Roux et al.(Roux et al., 2000). IL-6 and also M-CSF activate the expression of C/EBPβ factors (Akira et al., 1990; Komuro et al., 2003). Treatment of primary macrophages with IL-6 for 24 hours increased M-CSF transcription relative to untreated cells (50.1±0.4 and 13.0±0.6 RLU/μg of protein, respectively) and production (344.9±36.7 and 179.8±34.5 μg/ml, respectively). Although the effect of M-CSF on IL-6 production was previously known (Ji et al., 2000), to our knowledge, this is the first report showing that IL-6 up-regulates M-CSF. Since M-CSF has been reported to up-regulate IL-6 (Ji et al., 2000), the upregulation of M-CSF by IL-6 would thus form a positive feedback loop between M-CSF and IL-6.

### Identification of 4 C/EBPβ Binding Sites in the M-CSF Promoter

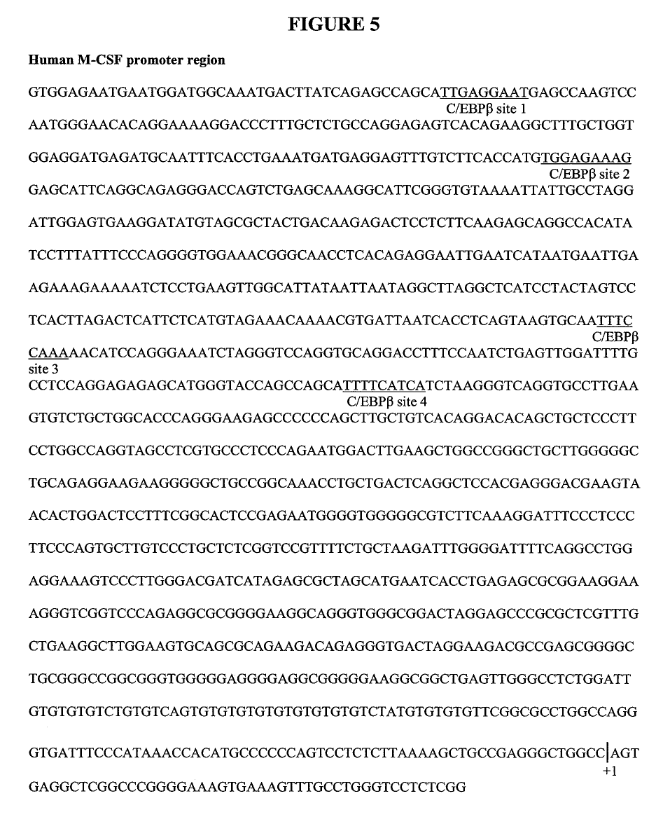

Four putative C/EBPβ binding sites were identified in the human M-CSF promoter using TF search (www.cbrc.jp/research/db/TFSEARCH.html) (threshold score of 84) (FIG. 5). The binding sites of transcription factors previously shown to interact with HIV-1 LTR promoter (Pereira et al., 2000) are also found in the M-CSF promoter, including NFκB (2 sites), AP-1 (4 sites), Sp-1 (2 sites), Oct-1 (3 sites), USF (4 sites) and IRF (1 site). Some of these sites have been previously reported (Ladner et al., 1987; Rajavashisth et al., 1995)

### LAP Up-Regulates M-CSF

To determine whether the activating isoform of C/EBPβ, LAP, is involved in M-CSF regulation, THP1 cells were co-transfected with a LAP expression vector and the pGL3-M-CSF construct. At 24 hours post-transfection, a 7-fold increase in M-CSF promoter activity by LAP was observed. In primary monocytes, a 2.5-fold increase in the M-CSF promoter activity and a 6-fold increase in M-CSF production were observed. Primary macrophages showed a 4-fold increase in M-CSF promoter activity and a 2-fold increase in M-CSF production by LAP (Table II

TABLE II
TABLE II. Effect of a LAP expression vector on the M-CSF promoter activity and the M-CSF
production in THP1 cells, primary human monocytes and primary human macrophages.

| | M-CSF promoter activity (RLU/Ug prot) | | M-CSF production (pg/ml) | |
|---|---|---|---|---|
| | pcDNA | pLAP | pcDNA | pLAP |
| THP1 | 29.2 ± 5.6 * | 207.2 ± 55.7 * | ND [symbol] | ND |
| Primary monocytes | 26 ± 8.1 [symbol] | 65.5 ± 11.7 [symbol] | 8.6 ± 4.8 * | 55.5 ± 3.9 * |
| Primary macrophages | 14.4 ± 2.2 [symbol] | 53.2 ± 9.2 [symbol] | 204.3 ± 20.9 [symbol] | 376.6 ± 39.5 [symbol] |

[symbol]

ND, not determined.
* p value < 0.01; [symbol]
, p value <0.05.

### C/EBPβ is Involved in Vpr-Dependent M-CSF Activation

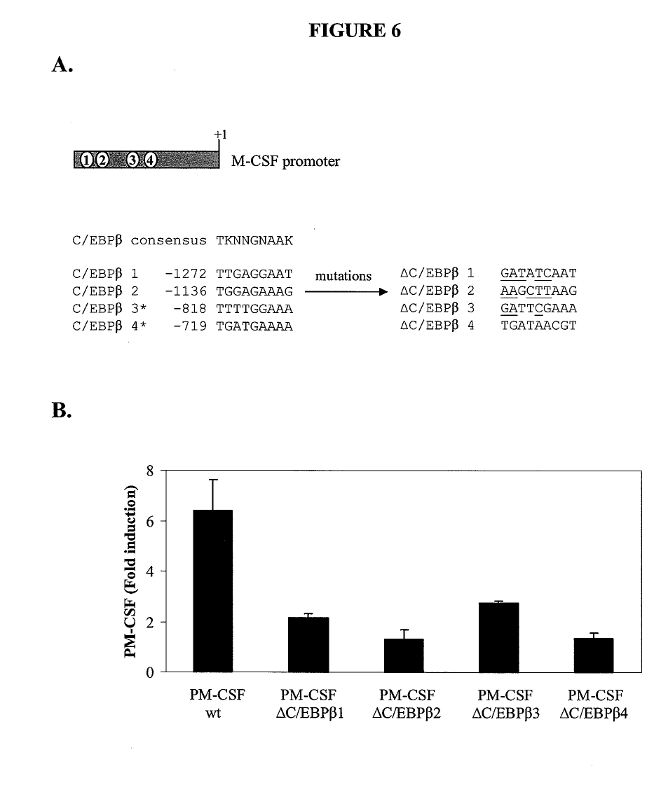

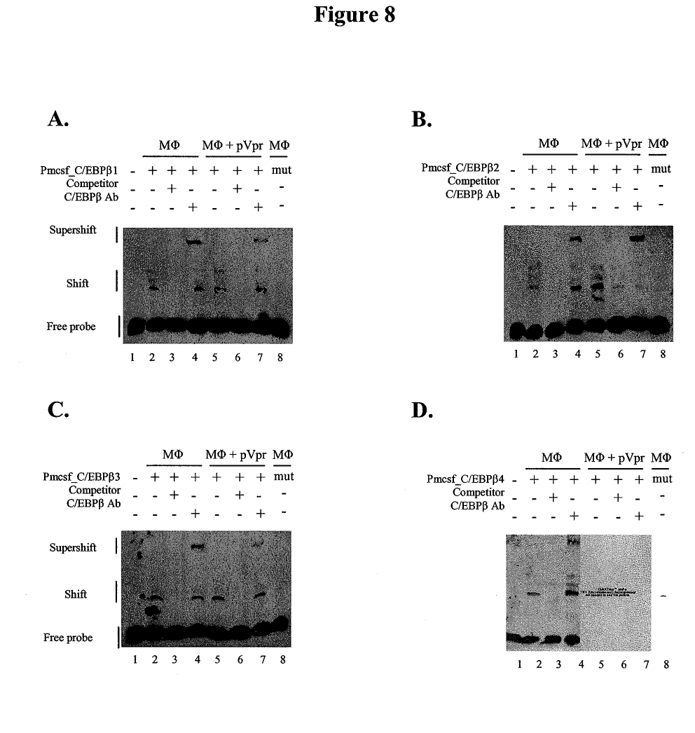

Liver-enriched transcriptional inhibitory protein (LIP) has been previously demonstrated to inhibit the transcriptional activity of LAP by forming heterodimers with LAP or by competing for DNA binding sites as a homodimer (Descombes and Schibler, 1991). To assess the effect of LIP on Vpr stimulated M-CSF expression, co-transfection experiments were performed in primary macrophages using both Vpr and LIP expression vectors (FIG. 6). Ectopic expression of LIP decreased Vpr-dependent M-CSF promoter activity as well as M-CSF production 24 hours post-transfection. Co-transfection of Vpr with an siRNA specific for C/EBPβ also decreased Vpr-dependent M-CSF production (FIG. 8). Western blot analyses clearly demonstrated that the siRNA efficiently decreases the level of C/EBPβ protein (FIG. 8). The neutralization of the Vpr-dependent effects on M-CSF by LIP or by siRNA directed against C/EBPβ supports the involvement of C/EBPβ factors in the M-CSF regulation mediated by Vpr.

To determine the specific C/EBPβ sites required for Vpr transactivation of M-CSF, site-directed mutants of individual C/EBPβ binding sites were generated in the pGL3-M-CSF construct. Each of the four mutated M-CSF promoters exhibited luciferase activity similar to the wild-type promoter in primary macrophages, suggesting that the deleted regions do not contribute significantly to the basal expression levels of M-CSF in macrophages (data not shown). In contrast, co-transfection of the mutant M-CSF promoter constructs with the Vpr expression plasmid in primary macrophages demonstrated that mutagenesis of each of the 4 C/EBPβ sites markedly decreases M-CSF promoter activity induced by Vpr (FIG. 5B). Thus, intact C/EBPβ sites in the M-CSF promoter are necessary for M-CSF production by Vpr. The mutated promoters retained inducibility by TNFα at levels similar to the wild-type promoter, as expected (data not shown). These results demonstrate functional C/EBPβ binding sites within the M-CSF promoter and their involvement in activation by HIV-1 Vpr.

### C/EBPβ Binds to the M-CSF Promoter

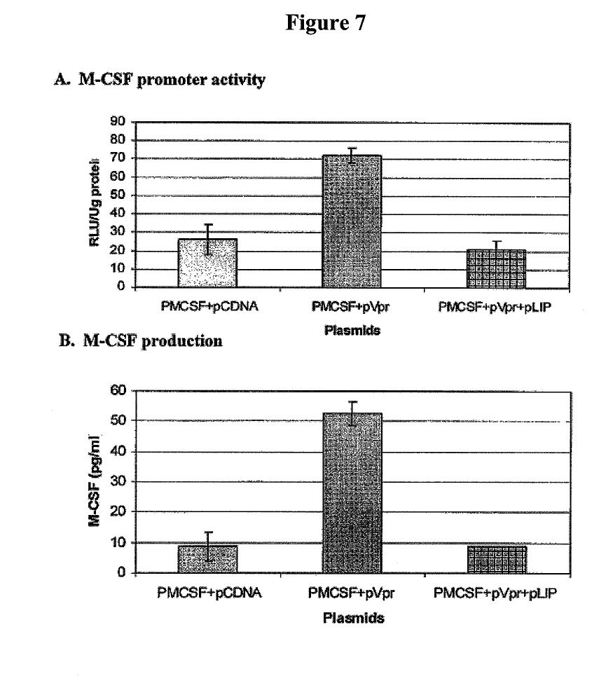

To determine the interaction of C/EBPβ with putative C/EBP binding sites within the M-CSF promoter, electrophoretic mobility shift assays (EMSA) were performed using double-stranded biotin-labeled oligonucleotide probes containing each of the 4 putative C/EBPβ binding sites from the M-CSF promoter region. Each of the four probes (Pmcsf_C/EBPβ1-4) are represented by panels A-D. Nuclear extracts prepared from untransfected and Vpr-transfected primary human macrophages were used in this assay (FIG. 7, Panels A-D). DNA-protein complexes were formed when each of the 4 probes were incubated with nuclear extracts from untransfected macrophages (lane 2, FIG. 7, Panels A-D). When extracts from Vpr-transfected macrophages were used, a band of the same migration and intensity was observed (lane 5, FIG. 7, Panels A-D). Protein binding to each of the 4 C/EBPβ probes was abolished by an excess of unlabeled probes carrying the same sequence, demonstrating the specificity of the interaction (lanes 3 and 6, FIG. 7, Panels A-D). Addition of anti-C/EBPβ antibody to the binding mixture induced a supershift of the specific DNA-protein complexes, confirming that C/EBPβ was present in these complexes (lanes 4 and 7, FIG. 7, Panels A-D). The formation of the complex was sequence specific since no complex was observed with the probes corresponding to the mutated sequences within each of the 4 C/EBPβ binding sites, showing that the mutated nucleotides are essential for C/EBPβ binding on the M-CSF promoter (lane 8, FIG. 7, Panels A-D).

### Vpr Increases the Phosphorylation of C/EBPβ

To investigate how Vpr activates M-CSF production via C/EBP factors, western blots were performed to examine possible alterations in C/EBPβ expression in untransfected and Vpr-transfected primary macrophages. These studies revealed similar expression of C/EBPβ with or without Vpr (FIG. 8). Since C/EBPβ factors are activated by phosphorylation (Nakajima et al., 1993), the level of phosphorylated C/EBPβ was examined using an antibody specific to the phosphorylated threonine 235 of C/EBPβ. This revealed that Vpr increased the phosphorylation of C/EBPβ in primary human macrophages (FIG. 8).

### Vpr-Mediated M-CSF Production Occurs via the Glucocorticoid Pathway

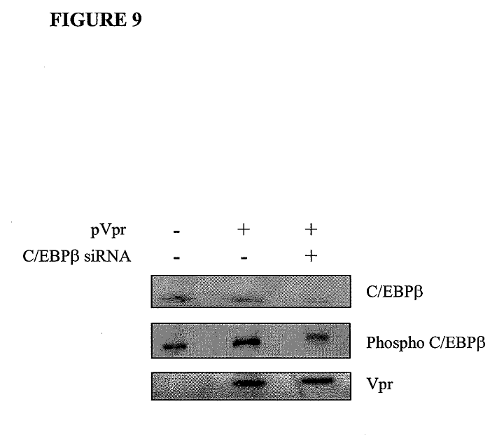

Several actions of Vpr, including inhibition of immune activation, apoptosis, cell proliferation, differentiation, maturation of macrophages, and viral replication have been linked to the glucocorticoid receptor pathway and the glucocorticoid antagonist RU486 (mifepristone) has been demonstrated to inhibit the various activities of Vpr(Kino et al., 1999; Muthumani et al., 2006; Ramanathan et al., 2002). Recently, Schafer et al. demonstrated that RU486 inhibits the Vpr-induced transactivation of the HIV-1 LTR as well as viral replication in PBMCs (Schafer et al., 2006). To determine the effect of RU486 on the Vpr-dependent M-CSF activation, primary human macrophages were co-transfected with pGL3-M-CSF and the Vpr expression vector and cultured with increasing concentrations of RU486 (FIG. 9). Cell lysates and supernatants collected at 1 or 2 days post-transfection showed a dose-dependent decrease in M-CSF promoter activity (FIG. 9A) and M-CSF production (FIG. 9B). At a concentration of 20 μM, RU486 inhibited the Vpr-dependent M-CSF promoter activity by greater than 50% (FIG. 9A). Similarly, Vpr induced M-CSF production was inhibited by greater than 50% at 10 μM; with a total inhibition of the Vpr effect at 50 μM. RU486 did not completely inhibit the basal level of M-CSF promoter activity (FIG. 9A) or basal level production (FIG. 9B) even at concentrations as high as 100 μM (in the absence of Vpr). It is worth noting that the concentration of RU486 required for inhibition of M-CSF induction by Vpr (approximately 10 μM) is similar to the concentration required to inhibit HIV-1 replication by CCR5 tropic virus infection of macrophages (Schafer et al., 2006).

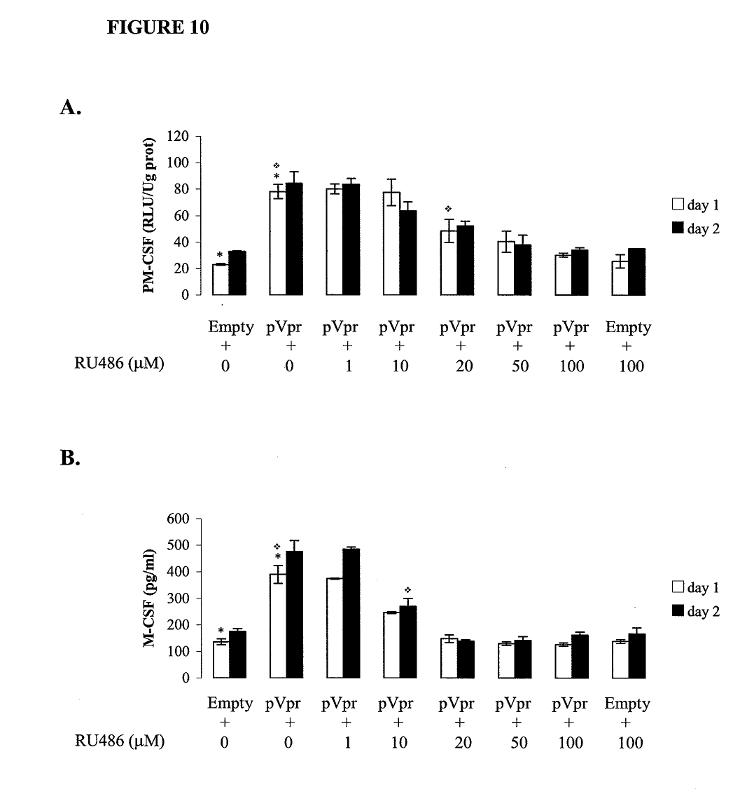

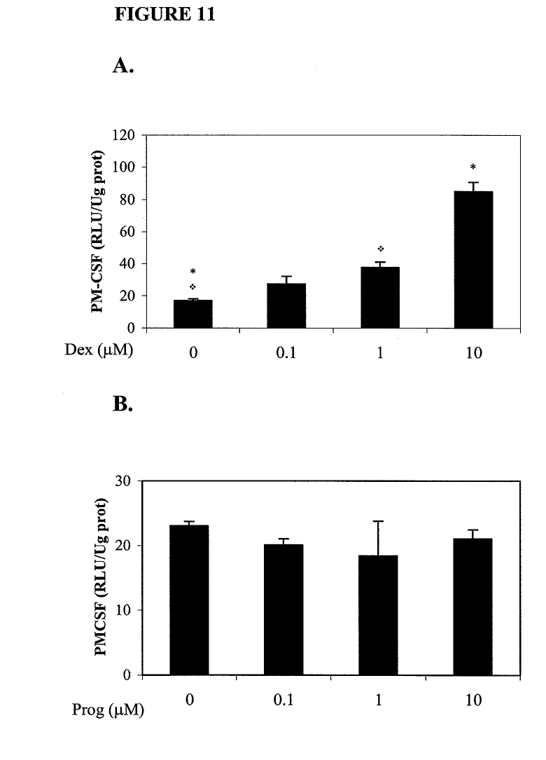

Considering C/EBPβ has also been demonstrated to be activated through the glucocorticoid receptor pathway (Savoldi et al., 1997) and our identification of C/EBPβ functional sites within the M-CSF promoter, the effect of dexamethasone on the M-CSF promoter was examined. Primary macrophages transfected with the pGL3-M-CSF construct in the presence of different concentrations of dexamethasone show an increase in M-CSF promoter activity (FIG. 10A). In contrast, no increase was observed after addition of progesterone (FIG. 10B). This suggests that M-CSF production occurs via the glucocorticoid but not progesterone receptor pathway in primary human macrophages.

Therapeutic strategies targeting M-CSF would likely reduce the number of suppressive macrophages and osteoclasts, restore DC number and function, and improve Th function by restoring the balance between MΦ-1 and MΦ-2 populations. Inhibiting M-CSF production, blocking the interaction of M-CSF with its receptor, cFMS and/or modulation of cFMS activity or expression could further impair HIV-1 replication in vivo, reduce the number of cell targets for infection, reduce the survival of HIV-1-infected reservoirs, reduce trafficking of macrophages and restore proper myeloid homeostasis. Thus, RU486 and C/EBPβ siRNA as inhibitors of M-CSF production are useful for the treatment of HIV-1 infection, and may have therapeutic value in other diseases where M-CSF has been implicated in pathogenesis (for reviews, see (Chitu and Stanley, 2006; Fixe and Praloran, 1998)). For example, M-CSF is up-regulated in patients with breast carcinoma and tumors of the reproductive tract (Kacinski, 1997) and may play a role in stimulating tumor progression and metastasis (Wyckoff et al., 2004). Recently, RU486 was demonstrated to prevent mammary tumorigenesis in Bcra1/p53-deficient mice (Poole et al., 2006). Furthermore, a positive correlation was found between macrophage numbers, adipocyte size and body mass, suggesting that M-CSF contributes to the development of obesity (Weisberg et al., 2003).

The inventors have demonstrated the central role of HIV-1 Vpr in M-CSF regulation. Vpr activates C/EBPβ factors by phosphorylation, which can then activate M-CSF transcription in the context of 4 C/EBPβ binding sites present in the M-CSF promoter. The modulation of Vpr-dependent M-CSF activation by agents is therefore a useful strategy for the treatment of HIV-1 infection/AIDS, and other diseases involving M-CSF dysregulation.

The present invention also includes a method of identifying a composition which inhibits activation of the M-CSF promoter. This method comprises the steps of: a) constructing a vector comprising a nucleic acid sequence encoding th M-CSF promoter region, for example SEQ ID NO: 22 and a nucleic acid sequence encoding a reporter molecule, the nucleic acid sequence encoding the reporter molecule being operably linked to the nucleic acid sequence encoding the sequence represented by SEQ ID NO: 22; b) introducing the vector into a host cell for a time and under conditions suitable for expression of the reporter gene; c) exposing the host cell to a composition which may inhibit activation of the M-CSF promoter and a substrate specific for the reporter molecule; and d) measuring the signal generated by reaction of the reporter molecule and the substrate in comparison to that produced by a control host cell, a smaller signal by the host cell of (c) indicating that the composition will modulate activation of the M-CSF promoter.

The identification of compounds which inhibit the M-CSF promoter activity and luciferase production may be carried out by the use of drug screening assays. Initially, a vector is created comprising an isolated DNA sequence encoding the promoter region of M-CSF, which is linked to the luciferase reporter gene. The vector may be, for example, a plasmid, a bacteriophage or a cosmid. The vector is then introduced into host cells under time and conditions suitable for activation of the M-CSF promoter. The host cells may be prokaryotic or eukaryotic cells. The host cells are then exposed to the test composition thought to block activation of the M-CSF promoter and luciferase gene expression. The cells are also exposed to a substrate for luciferase. One then measures the quantity of signals or light emitted from the luciferase-substrate reaction. If the amount of signals produced by the host cells, exposed to the composition in question, is lower than that produced by control cells (i.e., cells which have not been exposed to the composition), then the composition has inhibited the activity of the M-CSF promoter, and will be useful in inhibiting the expression of the M-CSF gene. If the amount of signals produced by the treated cells is equal to that produced by the control cells, the composition has not inhibited the activity of the M-CSF promoter and will not prevent M-CSF gene expression.

Once compositions have been identified which modulate the activity of the M-CSF promoter, such compositions may be administered to patients having any type of condition, for example, Acquired Immunodeficiency Syndrome (AIDS). The pharmaceutical composition may comprise a therapeutically effective amount of the inhibitor and an appropriate physiologically acceptable carrier (e.g., water, buffered water or saline). The dosage, form (e.g., suspension, tablet, capsule, etc.), and route of administration of the pharmaceutical composition (e.g., oral, topical, intravenous, subcutaneous, etc.) may be readily determined by a medical practitioner and may depend upon such factors as, for example, the patient's age, weight, immune status, and overall health.

Additionally, the present invention also encompasses compositions comprising antibodies derived using purified M-CSF, Vpr and/or C/EBPβ protein or a portion thereof which may be administered with, for example, an appropriate carrier (e.g., water, buffered water or saline). Subsequent to administration of the antibodies, they may bind to expressed M-CSF, Vpr and/or C/EBPb in the body in order to form a complex, thereby preventing the expressed M-CSF, Vpr and/or C/EBPb. The antibodies themselves, as well as portions thereof, are also encompassed within the scope of the present invention, as well as assays which comprise such antibodies or portions thereof.

It should be noted that the pharmaceutical compositions and antibodies may be utilized for veterinary applications or for agricultural applications. For example, the therapeutic composition may be administered to mammals such as, for example, horses, cows, sheep, goats, cats, dogs and pigs.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Gene activation” refers to any process that results in an increase in production of a gene product. A gene product can be either RNA (including, but not limited to, mRNA, rRNA, tRNA, and structural RNA) or protein. Accordingly, gene activation includes those processes that increase transcription of a gene and/or translation of a mRNA. Examples of gene activation processes that increase transcription include, but are not limited to, those that facilitate formation of a transcription initiation complex, those that increase transcription initiation rate, those that increase transcription elongation rate, those that increase processivity of transcription and those that relieve transcriptional repression (by, for example, blocking the binding of a transcriptional repressor). Gene activation can constitute, for example, inhibition of repression as well as stimulation of expression above an existing level. Examples of gene activation processes which increase translation include those that increase translational initiation, those that increase translational elongation and those that increase mRNA stability. In general, gene activation comprises any detectable increase in the production of a gene product, in some instances an increase in production of a gene product by about 2-fold, in other instances from about 2- to about 5-fold or any integer therebetween, in still other instances between about 5- and about 10-fold or any integer therebetween, in yet other instances between about 10- and about 20-fold or any integer therebetween, sometimes between about 20- and about 50-fold or any integer therebetween, in other instances between about 50- and about 100-fold or any integer therebetween, and in yet other instances between 100-fold or more.

“Gene repression” and “inhibition of gene expression” refer to any process which results in a decrease in production of a gene product. A gene product can be either RNA (including, but not limited to, MRNA, rRNA, tRNA, and structural RNA) or protein. Accordingly, gene repression includes those processes which decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes which decrease translation include those which decrease translational initiation, those which decrease translational elongation and those which decrease mRNA stability. Transcriptional repression includes both reversible and irreversible inactivation of gene transcription. In general, gene repression comprises any detectable decrease in the production of a gene product, in some instances a decrease in production of a gene product by about 2-fold, in other instances from about 2- to about 5-fold or any integer therebetween, in yet other instances between about 5- and about 10-fold or any integer therebetween, in still other instances between about 10- and about 20-fold or any integer therebetween, sometimes between about 20- and about 50-fold or any integer therebetween, in other instances between about 50- and about 100-fold or any integer therebetween, in still other instances 100-fold or more. In yet other instances, gene repression results in complete inhibition of gene expression, such that no gene product is detectable.

“Modulation” refers to a change in the level or magnitude of an activity or process. The change can be either an increase or a decrease. For example, modulation of gene expression includes both gene activation and gene repression. Modulation can be assayed by determining any parameter that is indirectly or directly affected by the expression of the target gene. Such parameters include, e.g., changes in RNA or protein levels, changes in protein activity, changes in product levels, changes in downstream gene expression, changes in reporter gene transcription (luciferase, CAT, β-galactosidase, β-glucuronidase, green fluorescent protein (see, e.g., Mistili & Spector, Nature Biotechnology 15:961-964 (1997)); changes in signal transduction, phosphorylation and dephosphorylation, receptor-ligand interactions, second messenger concentrations (e.g., cGMP, cAMP, IP3, and Ca2+), cell growth, and neovascularization. These assays can be in vitro, in vivo, and ex vivo. Such functional effects can be measured by any means known to those skilled in the art, e.g., measurement of RNA or protein levels, measurement of RNA stability, identification of downstream or reporter gene expression, e.g., via chemiluminescence, fluorescence, colorimetric reactions, antibody binding, inducible markers, ligand binding assays; changes in intracellular second messengers such as cGMP and inositol triphosphate (IP3); changes in intracellular calcium levels; cytokine release, and the like.

It will be appreciated that a wide variety of host cells and vectors can be used for the instant invention. The term “host cell” refers to one or more cells into which a recombinant DNA molecule is introduced. A “vector” is a replicon, such as a plasmid, phage, or cosmid, to which another DNA segment may be attached so as to bring about the replication of the attached segment. A DNA “coding sequence” or a “nucleotide sequence encoding” a particular protein, is a DNA sequence which is transcribed and translated into a polypeptide in vitro or in vivo when placed under the control of appropriate regulatory elements. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence.

DNA “control elements” refers collectively to promoters, ribosome binding sites, polyadenylation signals, transcription termination sequences, upstream regulatory domains, enhancers, IRES (“internal ribosomal entry site”) and the like, which collectively provide for the transcription and translation of a coding sequence in a host cell. Not all of these control sequences need always be present in a recombinant vector so long as the desired gene is capable of being transcribed and translated.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence. A control element, such as a promoter, “directs the transcription” of a coding sequence in a cell when RNA polymerase will bind the promoter and transcribe the coding sequence into mRNA, which is then translated into the polypeptide encoded by the coding sequence. Operably linked may also refer to an arrangement of two or more genes encoded on the same transcript. This arrangement results in the co-transcription of the genes, i.e., both genes are transcribed together since they are present on the same transcript. This operably linked arrangement of genes can be found in a naturally occurring DNA or constructed by genetic engineering. Further, according to this operable linkage, the genes can be translated as a polyprotein, i.e., translated as a fused polypeptide such that the resultant proteins are interlinked by a peptide bond from a single initiation event, or the genes can be translated separately from independent translation initiation signals, such as an IRES (“, which directs translation initiation of internally-situated open reading frames (i.e., protein-coding regions of a transcript).

Additionally, the present invention encompasses a vector comprising the above-described nucleic acid sequence and a nucleic acid sequence encoding a reporter molecule. The nucleic acid sequence encoding the reporter molecule is operably linked to an M-CSF promoter region set out in SEQ ID NO: 22, or a fragment, mutant, allele, derivative or variant thereof able to promote transcription, and optionally responsive to C/EBPβ. The reporter molecule may be selected from the group consisting of, for example, luciferase, β-galactosidase and Chloramphenicol Acetyltransferase (CAT). Preferably, the reporter molecule is luciferase. The present invention also includes a host cell comprising the above-described vector.

### Screening for Substances Affecting M-CSF Expression

The present invention provides the use of all or part of the nucleic acid sequence of the M-CSF promoter in methods of screening for substances which modulate the activity of the M-CSF promoter and increase or decrease the level of M-CSF expression. The invention provides for use of regions of the M-CSF promoter which are responsive to C/EBPβ. This invention also comprises compounds, compositions, and methods useful for modulating the expression and activity of other genes or proteins involved in pathways of M-CSF gene or promoter expression.

“Promoter activity” is used to refer to ability to initiate transcription. The level of promoter activity is quantifiable for instance by assessment of the amount of mRNA produced by transcription from the promoter or by assessment of the amount of protein product produced by translation of mRNA produced by transcription from the promoter. The amount of a specific mRNA present in an expression system may be determined for example using specific oligonucleotides which are able to hybridize with the mRNA and which are labeled or may be used in a specific amplification reaction such as the polymerase chain reaction. Use of a reporter gene facilitates determination of promoter activity by reference to protein production.

Further provided by the present invention is a nucleic acid construct comprising an M-CSF promoter region set out in SEQ ID NO: 22, or a fragment, mutant, allele, derivative or variant thereof able to promote transcription, and optionally responsive to C/EBPβ operably linked to a heterologous gene, e.g. a coding sequence. Generally, the gene may be transcribed into mRNA which may be translated into a peptide or polypeptide product which may be detected and preferably quantitated following expression. A gene whose encoded product may be assayed following expression is termed a “reporter gene”, i.e. a gene which “reports” on promoter activity.

The reporter gene preferably encodes an enzyme which catalyses a reaction which produces a detectable signal, preferably a visually detectable signal, such as a colored product. Many examples are known, including β-galactosidase and luciferase. β-galactosidase activity may be assayed by production of blue color on substrate, the assay being by eye or by use of a spectrophotometer to measure absorbance. Fluorescence, for example that produced as a result of luciferase activity, may be quantitated using a spectrophotometer. Radioactive assays may be used, for instance using chloramphenicol acetyltransferase, which may also be used in non-radioactive assays. The presence and/or amount of gene product resulting from expression from the reporter gene may be determined using a molecule able to bind the product, such as an antibody or fragment thereof. The binding molecule may be labeled directly or indirectly using any standard technique.

Those skilled in the art are well aware of a multitude of possible reporter genes and assay techniques which may be used to determine gene activity. Any suitable reporter/assay may be used and it should be appreciated that no particular choice is essential to or a limitation of the present invention.

The level of expression in the presence of the test substance may be compared with the level of expression in the absence of the test substance (i.e., a control level). A difference in expression in the presence of the test substance indicates ability of the substance to modulate gene expression. An increase in expression of the heterologous gene compared with expression of another gene not linked to a promoter as disclosed herein indicates specificity of the substance for modulation of the promoter.

A promoter construct may be introduced into a cell line using any technique previously described to produce a stable cell line containing the reporter construct integrated into the genome. The cells may be grown and incubated with test agents for varying times. The cells may be grown in 96 well plates to facilitate the analysis of large numbers of compounds. The cells may then be washed and the reporter gene expression analyzed. For some reporters, such as luciferase the cells will be lysed then analyzed.

Following identification of a substance which modulates or affects promoter activity, the substance may be investigated further. Furthermore, it may be manufactured and/or used in preparation, i.e. manufacture or formulation, of a composition such as a medicament, pharmaceutical composition or drug. These may be administered to individuals in need of such treatment.

siNA

This invention comprises compounds, compositions, and methods useful for modulating M-CSF gene or promoter expression using short interfering nucleic acid (siNA) molecules. This invention also comprises compounds, compositions, and methods useful for modulating the expression and activity of other genes involved in pathways of M-CSF gene or promoter expression and/or activity by RNA interference (RNAi) using small nucleic acid molecules. In particular, the instant invention features small nucleic acid molecules, such as short interfering nucleic acid (siNA), short interfering RNA (siRNA), double-stranded RNA (dsRNA), micro-RNA (miRNA), and short hairpin RNA (shRNA) molecules and methods used to modulate the expression or activity of M-CSF gene or promoter, or the activity or expression of other components of the M-CSF pathway.

A siNA of the invention can be unmodified or chemically-modified. A siNA of the instant invention can be chemically synthesized, expressed from a vector or enzymatically synthesized. The instant invention also features various chemically-modified synthetic short interfering nucleic acid (siNA) molecules capable of modulating M-CSF gene or promoter expression or activity in cells by RNA interference (RNAi). The use of chemically-modified siNA improves various properties of native siNA molecules through increased resistance to nuclease degradation in vivo and/or through improved cellular uptake. Further, contrary to earlier published studies, siNA having multiple chemical modifications retains its RNAi activity. The siNA molecules of the instant invention provide useful reagents and methods for a variety of therapeutic, diagnostic, target validation, genomic discovery, genetic engineering, and pharmacogenomic applications.

In one embodiment, the invention features a double-stranded short interfering nucleic acid (siNA) molecule that modulates expression of M-CSF gene or promoter, wherein said siNA molecule comprises about 19 to about 21 base pairs. In one embodiment, the invention features a siNA molecule that modulates expression of a M-CSF gene or promoter, for example, wherein the M-CSF gene or promoter comprises M-CSF encoding sequence. In one embodiment, the invention features a siNA molecule that down-regulates expression of a M-CSF gene or promoter, for example, wherein the M-CSF gene or promoter comprises M-CSF non-coding sequence or regulatory elements involved in M-CSF gene or promoter expression.

In one embodiment, the invention features one or more chemically-modified siNA constructs having specificity for M-CSF expressing nucleic acid molecules, such as RNA encoding a M-CSF protein. Non-limiting examples of such chemical modifications include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy a basic residue incorporation. These chemical modifications, when used in various siNA constructs, are shown to preserve RNAi activity in cells while at the same time, dramatically increasing the serum stability of these compounds. Furthermore, contrary to the data published by Parrish et al., supra, applicant demonstrates that multiple (greater than one) phosphorothioate substitutions are well-tolerated and confer substantial increases in serum stability for modified siNA constructs.

In one embodiment, a siNA molecule of the invention comprises modified nucleotides while maintaining the ability to mediate RNAi. The modified nucleotides can be used to improve in vitro or in vivo characteristics such as stability, activity, and/or bioavailability. For example, a siNA molecule of the invention can comprise modified nucleotides as a percentage of the total number of nucleotides present in the siNA molecule. As such, a siNA molecule of the invention can generally comprise about 5% to about 100% modified nucleotides (e.g., 5%, 10%, 15%, 20%, 25%, 30%, 35%, 40%, 45%, 50%, 55%, 60%, 65%, 70%, 75%, 80%, 85%, 90%, 95% or 100% modified nucleotides). The actual percentage of modified nucleotides present in a given siNA molecule will depend on the total number of nucleotides present in the siNA. If the siNA molecule is single stranded, the percent modification can be based upon the total number of nucleotides present in the single stranded siNA molecules. Likewise, if the siNA molecule is double stranded, the percent modification can be based upon the total number of nucleotides present in the sense strand, antisense strand, or both the sense and antisense strands.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6.times.sodium chloride/sodium citrate (SSC) at about 45° C., followed by two washes in 0.2×SSC, 0.1% SDS at least at 50° C. (the temperature of the washes can be increased to 55 C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6.times.SSC at about 45.degree. C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C.

### Test Agents

Test agents that can be screened with methods of the present invention include polypeptides, β-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, small molecules, siNA, siRNA, dsRNA, dsDNA, anti-senseDNA, nucleic acids, antibodies, polyclonal antibodies, monoclonal antibodies, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be natural occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of M-CSF, M-CSF promoter, C/EBPβ, and/or Vpr. A number of assays are available for such screening.

Libraries of test agents to be screened with the claimed methods can also be generated based on structural studies of M-CSF or its fragments. Such structural studies allow the identification of test agents that are more likely to bind to M-CSF. The three-dimensional structure of M-CSF or its fragments (e.g., its catalytic domain) can be studied in a number of ways, e.g., crystal structure and molecular modeling. Methods of studying protein structures using x-ray crystallography are well known in the literature. See Physical Bio-chemistry, (85-86). Computer modeling of a target protein (e.g., M-CSF, C/EBPβ, and/or Vpr) provides another means for designing test agents for screening modulators of the target protein. Methods of molecular modeling have been described in the literature, e.g., U.S. Pat. No.5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR).

Modulators of the present invention also include antibodies that specifically bind to M-CSF, M-CSF promoter, C/EBPβ, and/or Vpr. Such antibodies can be monoclonal or polyclonal. Such antibodies can be generated using methods well known in the art. For example, the production of non-human monoclonal antibodies, e.g., murine or rat, can be accomplished by, for example, immunizing the animal with M-CSF or its fragment. Such an immunogen can be obtained from a natural source, by peptides synthesis or by recombinant expression.

Humanized forms of mouse antibodies can be generated by linking the CDR regions of non-human antibodies to human constant regions by recombinant DNA techniques. e(90) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to M-CSF.

### Antibodies

Additionally, the present invention includes a purified antibody produced in response to immunization with Vpr, M-CSF or C/EBP [symbol] as well as a composition comprising this purified antibody.

Antibodies refers to single chain, two-chain, and multi-chain proteins and glycoproteins belonging to the classes of polyclonal, monoclonal, chimeric, and hetero immunoglobulins (monoclonal antibodies being preferred); it also includes synthetic and genetically engineered variants of these immunoglobulins. “Antibody fragment” includes Fab, Fab′, F(ab′)2, and Fv fragments, as well as any portion of an antibody having specificity toward a desired target epitope or epitopes. A humanized antibody is an antibody derived from a non-human antibody, typically murine, that retains or substantially retains the antigen-binding properties of the parent antibody but which is less immunogenic in humans (Jones et al., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994)).

### Expression Systems and Host Cells

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC®. 2.0 from INVITROGEN® and BACPACK®. BACULOVIRUS EXPRESSION SYSTEM FROM CLONTECH

Host cells of the invention include, but are not limited to, bacterial cells, such as any Gram-positive, such as Bacillus subtilis, or Gram-negative bacterium, such as Escherichia coli, or any other suitable bacterial strain, fungal cells, such as the yeast Saccharomyces cerevisiae, animal cells, such as hamster, human, or monkey, plant cells, such as Arabidopsis thaliana, or insect cells, such as mosquito, or any other suitable cell. Host cells can be unicellular, or can be grown in tissue culture as liquid cultures, monolayers or the like. Host cells may also be derived directly or indirectly from tissues, such as liver, blood, or skin cells. Vectors can include plasmids, such as pBluescript SK, pBR322, and pACYC 184, cosmids, or virus/bacteriophage, such as pox virus vectors, baculovirus vectors, adenovirus vectors, and lambda, and artificial chromosomes, such as yeast artificial chromosomes (YAC), P1-derived artificial chromosomes (PACs) and bacterial artificial chromosomes (BACs), so long as they are compatible with the host cell, i.e., are stably maintained and replicated. Further, preferred prokaryotic vectors include plasmids such as those capable of replication in E. coli, for example, pBR322, ColE1, pSC101, pACYC 184, etc. (see Maniatis et al., Molecular Cloning: A Laboratory Manuel), Bacillus plasmids such as pC194, pC221, pT127, etc. (Gryczan, T., The Molecular Biology of the Bacilli, Academic Press, NY (1982), pp. 307-329); Streptomyces plasmids including pIJ101 (Kendall, K. J. et al., (1987) J. Bacteriol. 169:4177-4183); Streptomyces bacteriophages such as phiC31 (Chater, K. F. et al., in: Sixth International Symposium on Actinomycetal es Biology, Akademiai Kaido, Budapest, Hungary (1986), pp. 45-54), and Pseudomonas plasmids (John, J. F., et al. (1986) Rev. Infect. Dis. 8:693-704), and Izaki, K (1978) Jpn. J. Bacteriol. 33:729-742). Preferred eukaryotic plasmids include BPV, vaccinia, SV40, 2-micron circle, etc., or their derivatives. Such plasmids are well known in the art (Botstein, D., et al. (1982) Miami Wint. Symp. 19:265-274; Broach, J. R., in: The Molecular Biology of the Yeast Saccharomyces: Life Cycle and Inheritance, Cold Spring Harbor Laboratory, Cold Spring Harbor, N.Y., pp. 445-470 (1981); Broach, J. R., (1982) Cell 28:203-204; Bollon, D. P., et al. (1980) J. Clin. Hematol. Oncol. 10:39-48; Maniatis, T., in: Cell Biology: A Comprehensive Treatise, Vol. 3: Gene Expression, Academic Press, N.Y., pp. 563-608 (1980)).

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

### Pharmaceutical Compositions and Administration

Administration of therapeutically effective amounts is by any of the routes normally used for introducing protein or encoding nucleic acids into ultimate contact with the tissue to be treated. The protein or encoding nucleic acids are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington's Pharmaceutical Sciences, 17.sup.th ed. 1985)).

The protein or encoding nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

In certain cases, alteration of a genomic sequence in a pluripotent cell (e.g., a hematopoietic stem cell) is desired. Methods for mobilization, enrichment and culture of hematopoietic stem cells are known in the art. See for example, U.S. Pat. Nos. 5,061,620; 5,681,559; 6,335,195; 6,645,489 and 6,667,064. Treated stem cells can be returned to a patient for treatment of various diseases including, but not limited to, SCID and sickle-cell anemia.

Nucleic Acids Encoding siNAs and Delivery to Cells

Conventional viral and non-viral based gene transfer methods can be used to introduce nucleic acids encoding engineered siNAs in cells (e.g., mammalian cells) and target tissues. Such methods can also be used to administer nucleic acids encoding siNAs to cells in vitro. In certain embodiments, nucleic acids encoding siNAs are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51 (1):31-44 (1995); Haddada et al., in Current Topics in Microbiology and Immunology Doerfler and Bohm (eds) (1995); and Yu et al., Gene Therapy 1:13-26 (1994).

Methods of non-viral delivery of nucleic acids encoding engineered siNAs include electroporation, lipofection, rnicroinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.) and BTX Molecular Delivery Systems (Holliston, Mass.).

Lipofection is described in e.g., U.S. Pat. No. 5,049,386, U.S. Pat. No. 4,946,787; and U.S. Pat. No.4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

In many gene therapy applications, it is desirable that the gene therapy vector be delivered with a high degree of specificity to a particular tissue type. Accordingly, a viral vector can be modified to have specificity for a given cell type by expressing a ligand with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a siNA comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a siNA nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Vectors (e.g., retroviruses, adenoviruses, liposomes, etc.) containing therapeutic siNA nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route. Methods for introduction of DNA into hematopoietic stem cells are disclosed, for example, in U.S. Pat. No. 5,928,638.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### Luciferase Reporter

The luciferase reporter construct may comprise a single luciferase coding region or multiple luciferase coding regions. The luciferase coding region may encode a luciferase product with destabilizing elements, such as Active Motif's RapidReporter® Gaussia luciferase assay which utilizes double destabilizing elements to degrade both the luciferase protein and mRNA. The dual luciferase assay is described in U.S. Pat. No. 6,143,502, incorporated herein by reference. The firefly luciferase gene (fluc) has been cloned behind the renilla luciferase gene (rluc) into an altered vector pRL-SV40 vector (Promega Corp., Madison, Wis.).

The invention will be illustrated in more detail with reference to the following Examples, but it should be understood that the present invention is not deemed to be limited thereto.

### EXAMPLES

Cell Preparations

Peripheral blood mononuclear cells (PBMC) were isolated from heparinized whole blood or buffy coats acquired from healthy seronegative blood donors by density-gradient centrifugation (Histopaque-1017, Sigma-Aldrich, St. Louis, Mo.). CD14+ monocytes were isolated from prepared PBMC using the Miltenyi AutoMACS system (Miltenyi Biotec, Auburn, Calif.). Briefly, cells were incubated with anti-CD14 monoclonal antibody-coated microbeads (Miltenyi Biotec, Auburn, Calif.) at 4° C. for 15 minutes and run on an AutoMacs separator. Routinely, we achieve approximately 95% purity and no detectable activation of CD14+ monocytes using this method, as demonstrated by flow cytometry. Recovered CD14+ monocytes were used immediately in transfection experiments. For macrophage preparation, PBMC were incubated in RPMI 1640 (Invitrogen, Carlsbad, Calif.) containing 20% FBS, 10% human AB serum, 2 mM L-glutamine and penicillin (50 U/ml)/streptomycin (50 μg/ml) overnight at 37° C. Adherent cells were washed 3 times with phosphate-buffered saline (PBS), removed with cold PBS 0.02% EDTA and differentiated into macrophages for 6 days in DMEM supplemented with 10% human AB serum and 2 mM L-glutamine. THP1 cells were cultivated in RPMI 1640 supplemented with 10% FBS, 2 mM L-glutamine and penicillin (50 U/ml)/streptomycin (50 μg/ml) (=complete medium).

Plasmids

The M-CSF promoter (−1310/+48 bp) was amplified from human genomic DNA by PCR with the primers 5′-GTGGAGAATGAATGGATGGCAAATGAC-3′ (SEQ ID NO: 20) and 5′-CCGAGAGGACC CAGGCAAACTTT-3′ (SEQ ID NO: 21). The PCR product was ligated into the pGL3-basic vector (Promega Corporation, Madison, Wis.) to form plasmid pGL3-M-CSF. Mutations in the putative C/EBPβ binding sites in the M-CSF promoter were generated by amplification of the whole plasmid pGL3-M-CSF using site-directed mutated primers listed in Table III (F/RpmcsfΔC/EBPβ1-4). Amplification consisted of 30 cycles of 30 s at 94° C., 30 s at 58° C., and 6 min at 72° C. All the constructs were verified by sequencing (Nucleic Acid Facility, Jefferson University, PA USA).

TABLE III

| | | |
|---|---|---|
| FPmcsf_C/EBPβ1 | 5′-GACTTATCAGAGCCAGCATTGAGGAATGAGCCAAGTCCAATGGG-3′ | (SEQ ID NO: 1) |
| RPmcsf_C/EBPβ1 | 5′-CCCATTGGACTTGGCTCATTCCTCAATGCTGGCTCTGATAAGTC-3′ | (SEQ ID NO: 2) |
| FPmcsf_C/EBPβ2 | 5′-GGAGTTTGTCTTCACCATGTGGAGAAAGGAGCATTCAGGCAGAGG-3′ | (SEQ ID NO: 3) |
| RPmcsf_C/EBPβ2 | 5′-CCTCTGCCTGAATGCTCCTTTCTCCACATGGTGAAGACAAACTCC-3′ | (SEQ ID NO: 4) |
| FPmcsf_C/EBPβ3 | 5′-CACCTCAGTAAGTGCAATTTCCAAAAACATCCAGGGAAATC-3′ | (SEQ ID NO: 5) |
| RPmcsf_C/EBPβ3 | 5′-GATTTCCCTGGATGTTTTTGGAAATTGCACTTACTGAGGTG-3′ | (SEQ ID NO: 6) |
| FPmcsf_C/EBPβ4 | 5′-GGGTACCAGCCAGCATTTTCATCATCTAAGGGTCAGGTGCC-3′ | (SEQ ID NO: 7) |
| Rpmcsf_C/EBPβ4 | 5′-GGCACCTGACCCTTAGATGATGAAAATGCTGGCTGGTACCC-3′ | (SEQ ID NO: 8) |
| FPmcsfΔC/EBPβ1 | 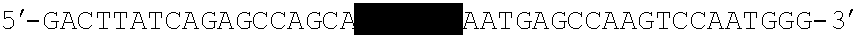 | (SEQ ID NO: 9) |
| RPmcsfΔC/EBPβ1 | 5′-CCCATTGGACTTGGCTCATTGATATCTGCTGGCTCTGATAAGTC-3′ | (SEQ ID NO: 10) |
| FPmcsfΔC/EBPβ2 | 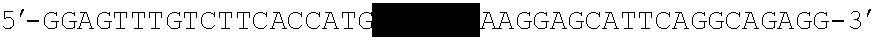 | (SEQ ID NO: 11) |
| RPmcsfΔC/EBPβ2 | 5′-CCTCTGCCTGAATGCTCCTTAAGCTTCATGGTGAAGACAAACTCC-3′ | (SEQ ID NO: 12) |
| FPmcsfΔC/EBPβ3 | 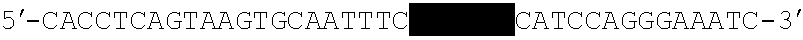 | (SEQ ID NO: 13) |
| RPmcsfΔCIEBPβ3 | 5′-GATTTCCCTGGATGGGATTCGAAATTGCACTTACTGAGGTG-3′ | (SEQ ID NO: 14) |
| FPmcsfΔC/EBPβ4 | 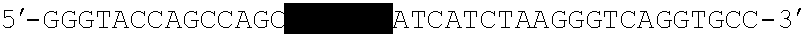 | (SEQ ID NO: 15) |
| RpmcsfΔC/EBPβ4 | 5′-GGCACCTGACCCTTAGATGATAAGCTTGCTGGCTGGTACCC-3′ | (SEQ ID NO: 16) |

Table III. Primers used for site-directed mutagenesis of the M-CSF promoter and EMSA^{a}
^{a}the restriction sites generated by the mutations (EcoR1 for ΔC/EBPβ1, HindIII for ΔC/EBPβ2 and 4, EcoRV for ΔC/EBPβ3) are in shadow.

LAP and LIP were generated by PCR amplification from human C/EBPβ cDNA (Openbiosystem, Human Verified Full-length cDNA Clones, MHS 1011, CA) utilizing forward primers for LAP, 5′-CACCATGGAAGTGGCCAACTTCTACTA-3′ (SEQ ID NO: 17), and LIP, 5′-CACCATGGCGGCGGGCTTCCCGTA-3′ (SEQ ID NO: 18), along with the reverse primer, 5′-CTAGCAGTGGCCGGAGGAGGCGAG-3′ (SEQ ID NO: 19) (Integrated DNA Technologies, Coralville, Iowa). The italicized nucleotides in the forward primer correspond to sequences necessary for directional cloning in the pcDNA3.1 TOPO vector (Invitrogen, San Diego, Calif.), while the underline portion corresponds to the respective start site of translation. The amplified LAP or LIP PCR product was ligated into the pcDNA3.1 TOPO vector as described by the manufacturer. Constructs were verified by sequencing (DNA Sequence Laboratory, Center for Molecular and Functional Genomics, Institute for Molecular Medicine and Infectious Disease, Drexel University College of Medicine). To confirm expression, 500 ng of LAP or LIP construct was transiently transfected into 3.0×107 293F cells using 293 fectin (Invitrogen) as described by the manufacturer and incubated for 24 hr. Western immunoblot analyses were performed on whole cell lysates using C/EBPβ antibody (C/EBPβ (C-19), Santa Cruz Biotech, Santa Cruz, Calif.) for detection of a 45 kD protein, LAP, or 20 kD protein, LIP.

The Vpr expression plasmid was previously described (Mahalingam et al., 1995). pNL4.3wt was obtained from NIH AIDS Research and Reference Reagent Program contributed by Dr. Malcolm Martin (Adachi et al., 1986) and pNL4.3Δvpr was a kind gift from Bassel Sawaya (Sawaya et al., 1999).

### Transfection and Luciferase Assay

THP1 cells (2×106/transfection) were transiently transfected with Effecten reagent (Qiagen, Santa Clara, Calif.) using 1 μg of plasmid DNA, 8 μl enhancer and 10 μl effectene. Cells were incubated in complete medium for 24 hours in 6-well plates. Human primary CD 14+ cells were transfected using the Human Monocyte Nucleofector Kit (Amaxa, Gaithersburg, Md.) according to the manufacturer's instructions. Briefly, 5×106 CD 14+ cells were resuspended in 100 μl of nucleofector solution (Amaxa) with 5 μg of DNA and electroporated using the program Y-001. Cells were then transferred in 1.5 ml of Human Monocyte Nucleofector medium (Amaxa) in 24-well plates for 24 hours. Human macrophages were transfected using the Human Macrophage Nucleofector Kit (Amaxa) according to the manufacturer's specifications. In brief, 0.5×106 macrophages were resuspended in 100 μl of nucleofector solution (Amaxa) with 5 μg of DNA and electroporated using the program Y-010. Cells were incubated for 24 hours in advanced RPMI (Invitrogen, Carlsbad, Calif.) supplemented with 10% FBS and 2 mM L-glutamine in 12-well plates. One day after transfection, cells were harvested by centrifugation and cell pellets were resuspended in 200 μl of lysis buffer (Promega). Twenty microliters of cell lysate was assayed for luciferase activity using the Luciferase Assay System (Promega). The protein concentration was determined with the BCA Protein Assay Reagent (Pierce, Rockford, Ill.) and the luciferase activity was expressed in Relative Light Unit (RLU) per μg of protein.

### Cytokine Assay

M-CSF and IL-6 production in cell culture supernatants were assessed by ELISA according to the manufacturer's instructions (R&D systems, Minneapolis, Minn.).

Small Interfering RNA (siRNA)

Gene silencing was performed with a siRNA specific for C/EBPβ (sc-29229, Santa Cruz). A non specific siRNA control (sc-37007, Santa Cruz) was utilized in parallel. Human primary macrophages were transfected with 1 μg of siRNA and 5 μg of DNA (2.5 μg of pGL3-M-CSF and 2.5 μg of the Vpr expression vector) using the Amaxa kit, as described above.

### Western Blot

Twenty-five μg of total extracts were heated at 95° C. for 5 min and separated on 12% SDS-polyacrylamide gel. After electrophoresis (1 h30 at 35 mA), proteins were transferred to a PVDF membrane (Hybond™-P, Amersham Biosciences, Piscataway, N.J.). The membrane was blocked in PBS-0.1% Tween-20 containing 5% non-fat milk one hour at room temperature, then incubated with a polyclonal antibody against C/EBPβ (1:50, sc-7962, Santa Cruz Biotechnology, Santa Cruz, Calif.), a phospho-C/EBPβ (Thr 235) antibody (1:500, Cell Signaling Technology, Beverly, Mass.) or a Vpr antibody (1:250) overnight at 4° C. Horseradish peroxidase-conjugated goat anti-rabbit IgG (sc-2030, Santa Cruz) or anti-mouse IgG2a-horseradish peroxidase-conjugated secondary antibody (sc-2061, Santa Cruz) was added at a dilution of 1:1000 for 1 h at room temperature. The membrane was developed by enhanced chemiluminescence with the Super-Signal substrate (Pierce, Rockford, Ill.).

### Electrophoretic Mobility Shift Assay (EMSA)

Human primary macrophages were transfected with 2 μg of the Vpr expression vector using the Amaxa kit, as described above. Nuclear extracts of Vpr-transfected or Vpr-untransfected macrophages were prepared using the NEPER nuclear and cytoplasmic extraction kit following the manufacturer's instructions (Pierce). The protein content was determined using the BCA Protein Assay Reagent (Pierce). Complementary oligonucleotides were 3′-biotinylated using the biotin 3′-end DNA labeling kit (Pierce) according to the manufacturer's instructions. The sequences of the oligonucleotides used are listed in Table III. After labeling, complementary strands were mixed together in an equimolar ratio and allowed to anneal to form double-stranded probe by incubating the primers for 5 min at 95° C. and slowly cooling down to room temperature. Binding reactions were carried out for 20 min at room temperature in the presence of 50 ng/μl poly(dI-dC), 0.05% Nonidet P-40, 5 mM MgCl2, 2.5% glycerol in 1×binding buffer (LightShift™ chemiluminescent EMSA kit, Pierce) using 20 fmol of biotin-end-labeled DNA and 5 μg of nuclear extract. Competition studies were performed by adding 4 pmol of unlabeled oligonucleotides. For Supershift analyses, 1 μg of an anti-C/EBPβ polyclonal antibody (sc-7962x, Santa Cruz Biotechnologies) was added to the reaction mixture for 30 minutes at 4° C. prior addition of probes. The reactions were loaded on 5% non-denaturing polyacrylamide gels and electrophoresed at 40 mA for 1 h in a 100 mM Tris-borate-EDTA buffer. The reactions were transferred to a positively charged nylon membrane (Hybond™-N+, Amersham). The biotin-labeled DNA was detected using horseradish peroxidase-conjugated streptavidin (LightShift™ chemiluminescent EMSA kit) according to the manufacturer's instructions.

### M-CSF Promoter Upstream of Luc

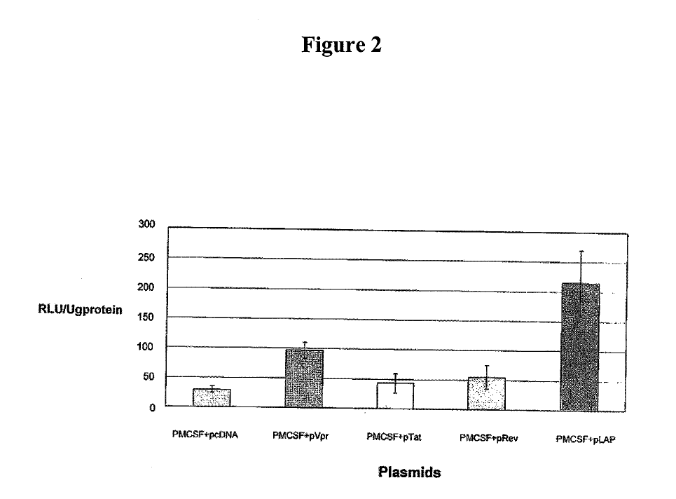

The M-CSF promoter was cloned upstream the luciferase reporter gene of the pGL3 basic vector (PMCSFpGL3). We first tested the potential effect of HIV-I proteins Vpr, Tat and Rev, and the large isoform of C/EBPβ, Liver-enriched transcriptional Activator Protein (LAP) on M-CSF promoter activity in THPI cells (FIG. 2). In the conditions tested, HIV-I Vpr and LAP upregulate M-CSF promoter activity by 3- and 7-fold, respectively; little or no effect is observed with HIV-I Tat or Rev.

### HI V-I Vpr on M-CSF Promoter Activity in Primary Human Monocytes

We next focused on the effect of HI V-I Vpr on M-CSF promoter activity in primary human monocytes in cotransfection study. As shown in FIG. 3A, a 3-fold increase in luciferase activity was observed in cells expressing Vpr compared to cells transfected with the empty vector (pcDNA). Culture supernatants were then analyzed for M-CSF production by ELISA (FIG. 3B). M-CSF levels were increased by at least 8-fold in human monocytes cotransfected with a Vpr expression vector. To investigate whether Liver-enriched transcriptional Inhibitory Protein, LIP (which inhibits the transcriptional activity of LAP) effects Vpr stimulation of M-CSF expression, we performed cotransfection experiments using both Vpr and LIP expression vectors. Ectopic expression of LIP decreases Vpr-dependent M-CSF promoter activity and MCSF production. The neutralization of the Vpr dependent effect on M-CSF by LIP supports the direct involvement of LAP in M-CSF regulation because LIP is a specific inhibitor. Thus, Vpr could activate M-CSF transcription/production by recruiting LAP to the M-CSF promoter.

### Statistical Analysis

Various transfection and treatment studies were compared by one-way analysis of variance (ANOVA) followed by Tukey-Kramer Multiple Comparisons post-test using Graph Pad Prism version 3.00 software, San Diego Calif. USA. P values less than 0.05 were considered to be statistically significant.

While the invention has been described in detail and with reference to specific examples thereof, it will be apparent to one skilled in the art that various changes and modifications can be made therein without departing from the spirit and scope thereof.

1. A nucleic acid comprising a reporter gene operatively linked to an M-CSF promoter region and the gene encoding HIV-1 Vpr.

2. The nucleic acid of claim 1, wherein the reporter gene is a luciferase gene.

3. The nucleic acid of claim 1, wherein the M-CSF promoter region is operatively linked to a CAAT enhancer binding protein site.

4. An isolated oligonucleotide comprising the M-CSF promoter region, wherein the MCSF promoter region is responsive to C/BPβ.

5. A reporter vector comprising the M-CSF promoter region, wherein the M-CSF promoter region is responsive to C/BPβ.

6. A host cell comprising a reporter gene operatively linked to an M-CSF promoter region and the gene encoding HIV-1 Vpr, wherein the M-CSF promoter region is responsive to C/BPβ.

7. The host cell of claim 6, wherein the host cell is a member selected from the group consisting of monocyte, macrophages, THP-1 cells, eukaryotic cells, and prokaryotic cells.

8. A cell line stably transfected with a reporter vector comprising the M-CSF promoter, wherein the M-CSF promoter region is responsive to C/BPβ.

9. A method of identification of compounds that modulate M-CSF promoter activity comprising:
(a) providing a reporter vector comprising a reporter gene and an M-CSF promoter region, wherein the M-CSF promoter region is responsive to C/BPβ;
(b) providing a test agent;
(c) providing C/BPβ;
(d) combining the reporter vector, the test agent, and C/BPβ;
(e) measuring reporter gene activity in the presence of test agent;
(f) measuring reporter gene activity in a control sample; and
(g) comparing reporter gene activity in the control sample compared to the test sample, to identify a compound which modulates M-CSF promoter activity.

10. The method of claim 9, wherein the reporter vector comprising a reporter gene and the M-CSF promoter region responsive to C/BPβ is in a stably transfected cell line.

11. A method of identifying a compound for the treatment of HIV-infection comprises determining the effect of the compound on HIV-1 Vpr-induced activation of the M-CSF promoter.

12. A method of identifying a potential HIV-1 therapeutic agent which inhibits Vpr induced activation of the M-CSF promoter comprising the steps of:
(a) providing a cell comprising a reporter gene operatively linked to an M-CSF promoter region;
(b) contacting the cell with a test agent in the presence of HIV-1 Vpr, wherein a decrement in the expression of the reporter gene in the presence of HIV-1 Vpr and the test agent, as compared to the expression of the reporter gene in the presence of Vpr and the absence of the test agent, indicates that the test agent is a potential HIV-1 therapeutic.

13. A method of identifying a potential HIV-1 therapeutic agent which inhibits HIV-1 Vpr-induced activation of the M-CSF promoter comprising the steps of:
(a) providing a cell comprising a reporter gene operatively linked to an M-CSF promoter region, and a vector comprising the gene encoding HIV-1 Vpr;
(b) contacting said cell with a test agent under conditions wherein said cells express Vpr, wherein a decrement in the expression of the reporter gene as compared to a control indicates that the test agent is a potential HIV-1 therapeutic.

14. The method of claim 13, wherein the cell is a member selected from the group consisting of monocytes, macrophages, THP-1 cells, eukaryotic cells, and prokaryotic cells.

15. The method of claim 13, wherein the M-CSF promoter is operatively linked to a CAAT enhancer binding protein site.

16. The method of claim 13, wherein the reporter gene is a luciferase gene.

17. A method of treating HIV infection in a patient in need thereof by administration of an effective amount of a glucocorticoid antagonist.

18. The method of claim 17, wherein the glucocorticoid antagonist is administered in an amount sufficient to have an effect selected from the group consisting of decreasing M-CSF levels in plasma, decreasing M-CSF levels in cerebrospinal fluid, decreasing M-CSF production by macrophages, and combinations thereof.

19. The method of claim 18, wherein the glucocorticoid antagonist is administered in combination with a compound selected from the group consisting of DHEA, antiretroviral drugs, M-CSF receptor antagonists, soluble M-CSF receptor molecules, and combinations thereof.

20. A method of treating a disease selected from the group consisting of malignancy, osteoporosis, autoimmune disorders, arthritis, obesity, and lipodystrophy in a patient in a need thereof by administration of a glucocorticoid antagonist in an amount sufficient to have an effect selected from the group consisting of decreasing M-CSF levels in plasma, decreasing M-CSF levels in cerebrospinal fluid, decreasing M-CSF production by macrophages, and combinations thereof.

21. The method of claim 20, wherein the glucocorticoid antagonist is administered in combination with a compound selected from the group consisting of DHEA, antiretroviral drugs, M-CSF receptor antagonists, soluble M-CSF receptor molecules, and combinations thereof.

22. The method of claim 20, wherein the glucocorticoid antagonist is RU486.

23. A method of treating HN infection in a patient in a need thereof by administration of an siRNA targeting HIV-1 Vpr-induced activation of the M-CSF promoter administered in an amount sufficient to have an effect selected from the group consisting of decreasing M-CSF levels in plasma, decreasing M -CSF levels in cerebrospinal fluid, decreasing M-CSF production by macrophages, and combinations thereof.

24. A method of treating HIV infection in a patient in a need thereof by administration of a glucocorticoid antagonist in an amount sufficient to have an effect selected from the group consisting of decreasing M-CSF levels in plasma, decreasing M-CSF levels in cerebrospinal fluid, decreasing M-CSF production by macrophages, and combinations thereof, in combination with an HIV antigen.

25. A method of treating HIV infection in a patient in a need thereof by administration of a glucocorticoid antagonist in an amount sufficient to have an effect selected from the group consisting of decreasing M-CSF levels in plasma, decreasing M-CSF levels in cerebrospinal fluid, decreasing M-CSF production by macrophages, and combinations thereof, after administration of an HIV antigen.

26. A method of preventing or treating HIV infection in a patient in a need thereof comprising: administering a preventive or prophylactic HIV vaccine; after exposure to the preventive or prophylactic HN vaccine, administering a glucocorticoid antagonist; whereby HIV infection is prevented or treated.

27. The method of claim 23, wherein the glucocorticoid antagonist is RU486.

28. A method of treating HIV infection in a patient in need thereof by administration of an effective amount of a compound which modulates activity of a member of the group selected from M-CSF promoter, M-CSF, Vpr, C/BPβ, and combinations thereof.

29. The method of claim 28, wherein the compound which modulates activity of a member of the group selected from M-CSF promoter, M-CSF, Vpr, C/BPβ, and combinations thereof is administered in an amount sufficient to have an effect selected from the group consisting of decreasing M-CSF levels in plasma, decreasing M-CSF levels in cerebrospinal fluid, decreasing M-CSF production by macrophages, and combinations thereof.

30. The method of claim 28, wherein the compound which modulates activity of a member of the group selected from M-CSF promoter, M-CSF, Vpr, C/BPβ, and combinations thereof is administered in combination with a compound selected from the group consisting of DHEA, antiretroviral drugs, M-CSF receptor antagonists, soluble M-CSF receptor molecules, and combinations thereof.

31. A method of treating a disease selected from the group consisting of malignancy, osteoporosis, autoimmune disorders, arthritis, obesity, and lipodystrophy in a patient in a need thereof by administration of a compound which modulates activity of a member of the group selected from M-CSF promoter, M-CSF, Vpr, C/BPβ, and combinations thereof in an amount sufficient to have an effect selected from the group consisting of decreasing M-CSF levels in plasma, decreasing M-CSF levels in cerebrospinal fluid, decreasing M-CSF production by macrophages, and combinations thereof.

32. The method of claim 31, wherein the compound which modulates activity of a member of the group selected from M-CSF promoter, M-CSF, Vpr, C/BPβ, and combinations thereof is administered in combination with a compound selected from the group consisting of DHEA, antiretroviral drugs, M-CSF receptor antagonists, soluble M-CSF receptor molecules, and combinations thereof.

33. The method of claim 31, wherein the compound modulates activity of a member of the group selected from M-CSF promoter, M-CSF, Vpr, C/BPβ, and combinations thereof is RU486.

34. An assay kit comprising a reporter vector comprising the M-CSF promoter, wherein the M-CSF promoter is responsive to C/BPβ.

35. The method of claim 24, wherein the glucocorticoid antagonist is RU486.

36. The method of claim 25, wherein the glucocorticoid antagonist is RU486.

37. The method of claim 26, wherein the glucocorticoid antagonist is RU486.

38. The method of claim 21, wherein the glucocorticoid antagonist is RU486.

39. The method of claim 32, wherein the compound modulates activity of a member of the group selected from M-CSF promoter, M-CSF, Vpr, C/BPβ, and combinations thereof is RU486.
